(19) United States
(12) Patent Application Publication
(10) Pub. No.: US 2019/0016048 A1
(43) Pub. Date: Jan. 17, 2019

(54) SYSTEMS AND METHODS FOR DESIGNING AND MANUFACTURING MULTI-COMPARTMENT CAPSULES

(71) Applicant: Multiply Labs Inc., San Francisco, CA (US)

(72) Inventors: Federico Parietti, San Francisco, CA (US); Kameron C. Chan, San Francisco, CA (US); Lawrence Zachary Bright, San Francisco, CA (US); Jeffrey Ackerman Cuhran, Warwick, RI (US); Alice Melocchi, Dalmine (IT); Lucia Zema, Milano (IT); Andrea Gazzaniga, Milano (IT); Joseph Siener Wilson, San Francisco, CA (US); Tiffany Amy Kuo, Daly City, CA (US); Vaibhav Sharma, Oakland, CA (US); Andrew Rutter, Oakland, CA (US)

(21) Appl. No.: 16/011,520

(22) Filed: Jun. 18, 2018

Related U.S. Application Data
(60) Provisional application No. 62/521,056, filed on Jun. 16, 2017.

(51) Int. Cl. B29C 64/209; B29C 64/118; B33Y 30/00; A61K 9/48; B01J 13/04

(52) U.S. Cl. CPC B29C 64/209; B29C 64/118; B01J 13/04; A61K 9/4808; A61K 9/48; B33Y 30/00

(57) ABSTRACT

A dispensing system includes a filament extruder that lays filament solutions. The filament extruder comprises multiple extruder sets. Each extruder set includes a first and a second extruder. Each of the first and the second extruder includes an extruder portion, a hot end portion, and a shroud. The extruder portion includes a first and a second insert that combine to form a channel and a needle tube that receives the filament solution from the channel and lays the filament solution on the target surface.

### TECHNICAL FIELD

The present disclosure relates generally to systems and methods for designing and manufacturing multi-compartment capsules, and, more particularly, a dispensing system, a filament extruder, a capsule, and a user interface.

### BACKGROUND

In general, a dispenser is configured to accurately and precisely dispense target doses of materials such as solids, liquids, and powders. The dispensing of powders using conventional dispensers incurs unique challenges because the bulk density of the material varies. This creates non-uniform flow which requires agitation, and the powder can often be lodged between moving parts in the dispenser due to the fine grain size of the material. Another challenge with dispensers is that the agitation necessary is typically provided by a vibration motor. These vibration motors have drawbacks in that that micro-impacts and galling occur between moving parts, which causes an increase in dispenser maintenance and costs.

Contamination of dispensing material is a concern for conventional dispensers. One source of contamination arises from the actuators in conventional dispensers. In such dispensers, these actuators are disposed above the dispensing material, are in close proximity to the dispensing material, and require lubrication. Accordingly, a leak in such lubrication creates a high risk of contamination.

Another drawback with conventional dispensers is that they require a large number of actuators when more than one material is dispensed. This renders the conventional dispenser susceptible to frequent maintenance.

Furthermore, conventional dispensers are disadvantageous because they are typically difficult to dismantle, clean, and reassemble. Despite this, thorough cleaning is necessary when a variety of dispensing materials are used by a single dispenser in order to avoid cross contamination. Thus, in conventional processes using conventional dispensers, it is necessary to laboriously disassemble and reassemble the dispensers for cleaning purposes.

Due to the above drawbacks, the performance and efficiency of conventional dispenser systems are unsatisfactory at best, decreases over time, and requires excessive maintenance and cleaning. This reduces overall manufacturing time, thereby increasing the costs of production.

Conventional three-dimensional printers lay a filament solution lay on a target surface using a filament extruder. Historically, three-dimensional printers have had a one-to-one relationship per filament extruder, meaning that each three-dimensional printer produced one product at a time using one extruder, which naturally limits throughput. Moreover, a three-dimensional printer that utilized multiple extruders to produce a single product that includes a variety of materials utilizes independent systems to operate each extruder. Cleaning of these printers is difficult and cumbersome.

Due to the serial nature of the three-dimensional printing process, products having complex or irregular shapes, and even interlocking parts, are formed. Also, products otherwise deemed difficult, or impossible, to fabricate using conventional methods may be designed.

Conventional three-dimensional printing machines have not been sufficiently utilized for the medical supplement manufacturing industry because they are difficult to clean. Refilling, replacing and or changing the filament in conventional three-dimensional printing machines often spreads contamination throughout the machine, requiring additional cleaning and maintenance. Heated portions of the extruder cannot be disassembled, and other portions that contact the filament are not easily removable, replaceable, and cleanable. Extended use of these parts eventually produces residue, which contaminates products. This contamination makes conventional three-dimensional printers particularly unsatisfactory for the medical supplement industry, where cleanliness is of the utmost concern. Moreover, a majority of the filament loading and unloading processes, as well as cleaning processes, associated with three-dimensional printers involves dismantling the entire printer, which halts manufacturing and increases overall costs.

While conventional three-dimensional printing machines typically have allowed manual changeover from one filament to another and associated cleaning procedures, such changeovers are not satisfactorily rapid and they are not satisfactorily easy to perform.

Thus, prior to the present disclosure there existed a need for a filament extruder provision for easier changeover of the three-dimensional printer from one filament to another and ease of cleaning.

Oral administration is one of the most prevalent methods for delivering active ingredients or medicaments to the body. Active ingredients or medicaments may be orally administered in a variety of physical states such as solid, liquid, or powder. Capsules have become the preferred drug delivery systems (DDS) for administering oral dosages.

Conventional capsules include a first compartment section, known as a base, and a second compartment section referred to as a cap. The two compartments of the capsule are designed so that the material to be encapsulated is dispensed into the base, and the open end of the cap section is correspondingly disposed over the open end of the base. The walls of the cap and base are in physical contact with one another forming a single internal compartment. A means for structurally sealing the cap in relation to the base is also incorporated into the manufacturing of capsules, thereby preventing contamination of the capsule. Moreover, conventional methods of producing capsules lack the means to create complex shapes such as an internal grid of extruded material and through holes that reduce in diameter as closer to an external surface of a capsule.

Advances in pharmacological therapy are achieved through the discovery of new molecules or the identification of more efficient methods of administration, e.g. the development of DDS. DDS exploits technological features, including design, composition, and manufacturing processes, to determine, modulate, and improve the drug availability at the site of action. In addition to the therapeutic advantages, DDS affords improvements in bioavailability, efficacy and compliance, as well as overall drug dose and side effect reduction. As such, the economic and health benefits related to the reduction and control of development costs and line extension through these improvements are important to the success of this technology.

A large number of encapsulates (carriers) used for DDSs are based on pharma-grade polymeric materials with a distinguishing behavior in the biological environment, including pH-dependent solubility, enzyme degradability, swelling (glassy to rubbery transition), and successive erosion and dissolution in aqueous fluids, bio-adhesion, and permeability. In some instances, the conventional DDS design strategy makes use of polymers inter-dispersed with the active ingredient in the form of a matrix system. In other instances, the conventional DDS design strategy applies the pharma-grade polymeric materials as a coating barrier onto active ingredient containing cores such as reservoir systems and osmotic pumps. In particular, for the coated systems, physical characteristics of the manufactured DDS pill, such as shape, dimension, and surface properties, as well as technological characteristics of thermal and mechanical resistance, friability, wettability, disintegration and dissolution tendency, and further stability characteristics of the inner core may impair or constrain the coating process and decrease on the system performance. Given the importance of DDS, new systems and methods for supporting DDS are needed in the art.

When designing a medicament and supplement plan, a user applying conventional art systems and methods often administers dosages intermittently throughout a time period, typically a day. The user is responsible for the physical dosage throughout the day, and must remember administration of the dosages at various time periods. Often the user forgets to administer the dosage at the required time, or is inconvenienced with carrying multiple dosages of multiple medicaments and supplements. Thus, there is a need for a user interface (UI) which alieves the problems of the conventional art which provides a means for the user to create a custom, single dosage capsule.

Given the above background, improvements regarding a dispensing system, a filament extruder, a capsule, and a user interface are needed in the art.

The information disclosed in this Background section is only for enhancement of understanding of the general background of the invention and should not be taken as an acknowledgement or any form of suggestion that this information forms the prior art already known to a person skilled in the art.

### SUMMARY

The dispensers, filament extruders, capsules, and user interfaces detailed in the present disclosure address the shortcomings in the prior art detailed above.

Various aspects of the present disclosure are directed to providing a dispensing system, which is configured to accurately and precisely dispense a target dose of material with minimal actuators, no moving parts in contact with the dispensed material, and designed for simplicity, less maintenance, failure modes, and contamination, as well as easier cleaning.

In accordance with an aspect of the present disclosure, the above and other objects can be accomplished by the provision of a dispensing system including: a dispenser, a fixing plate, a dispensing station, and a dial.

In some embodiments, the dispenser comprises a vibrating assembly that includes a hopper including one or more metering holes on a bottom surface thereof. The dispenser further comprises an upper base with a second insertion hole for receiving the stirrer and a stirrer disposed inside the hopper and fixed to the upper base. The dispenser further comprises a primary base with a plurality of spacers, a plurality of spring-dampers, a vibration device, a first insertion hole for receiving the hopper and a hopper hub, a pair of linear guides, and a first end of a gate spring. The dispenser further comprises a lower base in which a transfer block, gate, a second end of the gate spring, and gate hub are disposed.

The fixing plate is isolated from the vibrating assembly using the plurality of spring-dampers, and is configured to fix the dispenser to the dispensing station or the dial.

In some embodiments, the dispensing station comprises a stationary base, an upper base, a platform, a drive wheel to rotate the hopper, a first actuator configured to orient the upper base and a desired dispenser, a second actuator configured to engage the drive wheel with the hopper, a third actuator configured to rotate the drive wheel, and a fourth actuator configured to engage a transfer block of the dispenser.

In some embodiments, the dispensing system comprises a containment system and a support structure configured to contain a dispensed material and determine the weight of a dispensed material from the dispenser apparatus. The containment system comprise a primary base formed with a plurality of holes thereby allowing a plurality of prongs of the support structure to penetrate through. A bin is disposed on a top surface of the primary base and configured to accommodate a screen which filters material. A first fan is disposed below the bin and configured to draw air through the screen. A printing plate is disposed above the bin and formed in a ‘T’-shape, configured to be a target dispensing location. The support structure comprises a balance, which determines the mass of the dispensed material, and the plurality of prongs.

The dispensing system according to an exemplary embodiment of the present disclosure is provided to cure the drawbacks of the prior art while having the advantage of minimal moving parts and actuators to reduce maintenance and cleaning. In such embodiments, the actuators are distanced from the dispenser to prevent contamination. Further, a modular dispenser is provided that includes a plurality of dispensers that advantageously can be employed to dispense multiple materials.

Another aspect of the present disclosure is directed to providing a filament extruder that is easily assembled and disassembled for cleaning and maintenance. In some embodiments, the filament extruder is configured to produce a plurality of identical products utilizing a single three-dimensional printer. Moreover, in such embodiments the filament extruder has a limited heated volume and is capable of dispensing pharmaceutical grade material.

In accordance with another aspect of the present disclosure, the above and other objects are accomplished by the provision of a filament extruder that includes a plurality of extruders that each extruder a corresponding filament. Each extruder includes an extruder portion and a hot end portion.

In some embodiments, the extruder portion comprises a first plate and a second plate removeably coupled to the first plate. In such embodiments, the coupled first plate and second plate form a housing. A first insert is attached to the first plate, and a second insert is attached to the second plate. Moreover, the first insert and the second insert form a channel. A needle tube is in thorough communication with the channel to allow the filament to traverse through the extruder. The extruder portion further comprises a pinch wheel proximate to a first side of a first opening of the channel, a bearing proximate to a second side of the first opening of the channel, a lever coupled to the bearing, and a spring that actuates the lever.

In some embodiments, the hot end portion comprises, a heater block encompassing at least a portion of the needle tube, a heater configured to heat the heater block, and a thermistor configured as a detector of a temperature of a filament. In some embodiments, a shroud houses the hot end portion.

The filament extruder according to an exemplary embodiment of the present disclosure is provided to cure the disadvantages of the prior art while having the advantages of being easily assembled and disassembled for cleaning and maintenance while printing a plurality of identical products using one three-dimensional printer. The disclosed filament extruder has a limited heated volume, and is capable of dispensing pharmaceutical grade material.

Further aspects of the present disclosure are directed to providing a method of making a capsule comprising a plurality of compartments. In the method, the capsule is formed through the process of initializing extrusion of a filament solution through a filament extruder. The method further comprises laying the filament solution to form a base of the capsule and forming a first compartment in the plurality of compartments. The forming of the first compartment in the plurality of compartments creates a first barrier wall comprises a first predetermined release time. The method further comprises forming a second compartment in the plurality of compartments. The forming of the second compartment in the plurality of compartments creates a second barrier wall having a first predetermined release time. The method further comprises filling the first compartment with a first material and filling the second compartment with a second material. Moreover, the method further comprises sealing the plurality of compartments thereby forming the capsule with a first sealed compartment and a second sealed compartment. In some embodiments, each compartment in the plurality of compartments is formed, filled, then sealed before a proceeding compartment in the plurality of compartments may be formed.

The disclosed methods for making a capsule that has a plurality of compartments advantageously cures the disadvantages of the prior art while having the advantages of a plurality of compartments that are independently filled with one or more materials. The discloses capsules thereby impart predetermined release (performance) metrics in accordance with engineered capsule compartment wall thickness, composition, and overall design of the capsule.

Another aspect of the present disclosure is directed to providing a user interface, that enables a user (e.g., lab professional, medical care provider, pharmacist, vitamin provides, etc.) to custom design a capsule and the release (performance) metrics of each compartment in the capsule. The user interface invokes a questionnaire in electronic format on an interne enabled device. The questionnaire includes a plurality of questions including one or more biometric parameters associated with the target user (e.g., patient), one or more physician recommendations associated with the target user, one or more goals associated with the target user, and/or one or more nutritional requirements associated with the target user. In accordance with this aspect of the present disclosure a plurality of responses is received form the questionnaire posed to a user. The plurality of responses is stored in the interne enabled device. Responsive to a capsule request from the user, an end product is a designed in the form of a personalized capsule responsive to the plurality of responses to the questionnaire. This designing determines a modified-release schedule and an external capsule shape based upon all or a portion of the plurality of responses.

The user interface according to an exemplary embodiment of the present disclosure is provided to cure the disadvantages of the prior art while having the advantages of a means for the user to create a custom, single dosage capsule tailored to the user or to a patient or customer of the user.

The methods and apparatuses of the present disclosure have other features and advantages which will be apparent from or are set forth in more detail in the accompanying drawings, which are incorporated herein, and the following Detailed Description, which together serve to explain certain principles of the present invention.

### BRIEF DESCRIPTION OF THE DRAWINGS

FIG. 1 is an exploded view of the dispenser and fixing plate according to an exemplary embodiment of the present disclosure;

FIG. 2 is a view of the dispenser and fixing plate according to an exemplary embodiment of the present disclosure;

FIG. 3 is another view of the dispenser and fixing plate according to an exemplary embodiment of the present disclosure;

FIG. 4 is a sectional view of the dispenser and fixing plate according to an exemplary embodiment of the present disclosure from the view of FIG. 3;

FIG. 5 is a view of the hopper, gate, metering holes, and stirrer according to an exemplary embodiment of the present disclosure;

FIG. 6 is an illustration of the hopper, gate, and stirrer in the OFF position in accordance with an embodiment of the present disclosure;

FIG. 7 is an illustration of the hopper, gate, and stirrer in the ON position in accordance with an embodiment of the present disclosure;

FIG. 8 is a view of the dispensing station according to an exemplary embodiment of the present disclosure;

FIG. 9 is another view of the dispensing station according to an exemplary embodiment of the present disclosure;

FIG. 10 is an exploded view of the stationary base of the dispensing station according to an exemplary embodiment of the present disclosure;

FIG. 11 is an exploded view of the platform and the upper base of the dispensing station according to an exemplary embodiment of the present disclosure;

FIG. 12 is a view of the dispenser and dispensing station with the drive wheel disengaged according to an exemplary embodiment of the present disclosure;

FIG. 13 is a view of the dispenser and dispensing station with the drive wheel engaged according to an exemplary embodiment of the present disclosure;

FIG. 14 is a view of the dispenser, fixing plate, dispensing station, and dial;

FIG. 15 is another view of the dispenser, fixing, dispensing station, and dial;

FIG. 16 is a view of the fixing plate and notches according to an exemplary embodiment of the present disclosure;

FIG. 17 is a view of the dial and pegs according to an exemplary embodiment of the present disclosure

FIG. 18 is a view of the fixing plate and dial in the correct position according to an exemplary embodiment of the present disclosure;

FIG. 19 is a view of the fixing plate and dial in the incorrect position according to an exemplary embodiment of the present disclosure;

FIG. 20 is a view of the containment system according to an exemplary embodiment of the present disclosure;

FIG. 21 is a view of the containment system according to an exemplary embodiment of the present disclosure;

FIG. 22 is a plan view of the containment system according to an exemplary embodiment of the present disclosure;

FIG. 23 is a view of the support prongs and the printing plate engaged according to an exemplary embodiment of the present disclosure;

FIG. 24 is a view of the support prongs and the printing plate disengaged according to an exemplary embodiment of the present disclosure;

FIG. 25 is a view of the containment system and filament extruder according to an exemplary embodiment of the present disclosure;

FIG. 26 is a view of the dispenser and docking station according to an exemplary embodiment of the present disclosure;

FIG. 27 is a view of the containment system and filament extruder according to an exemplary embodiment of the present disclosure;

FIG. 28 is view of a filament extruder, in accordance with an embodiment of the present disclosure;

FIG. 29 is a partially exploded view of an extruder portion, in accordance with an embodiment of the present disclosure;

FIG. 30 is a sectional view of an extruder portion, in accordance with an embodiment of the present disclosure;

FIG. 31 is a view of a hot end portion, in accordance with an embodiment of the present disclosure;

FIG. 32 is a view of another hot end portion, in accordance with an embodiment of the present disclosure;

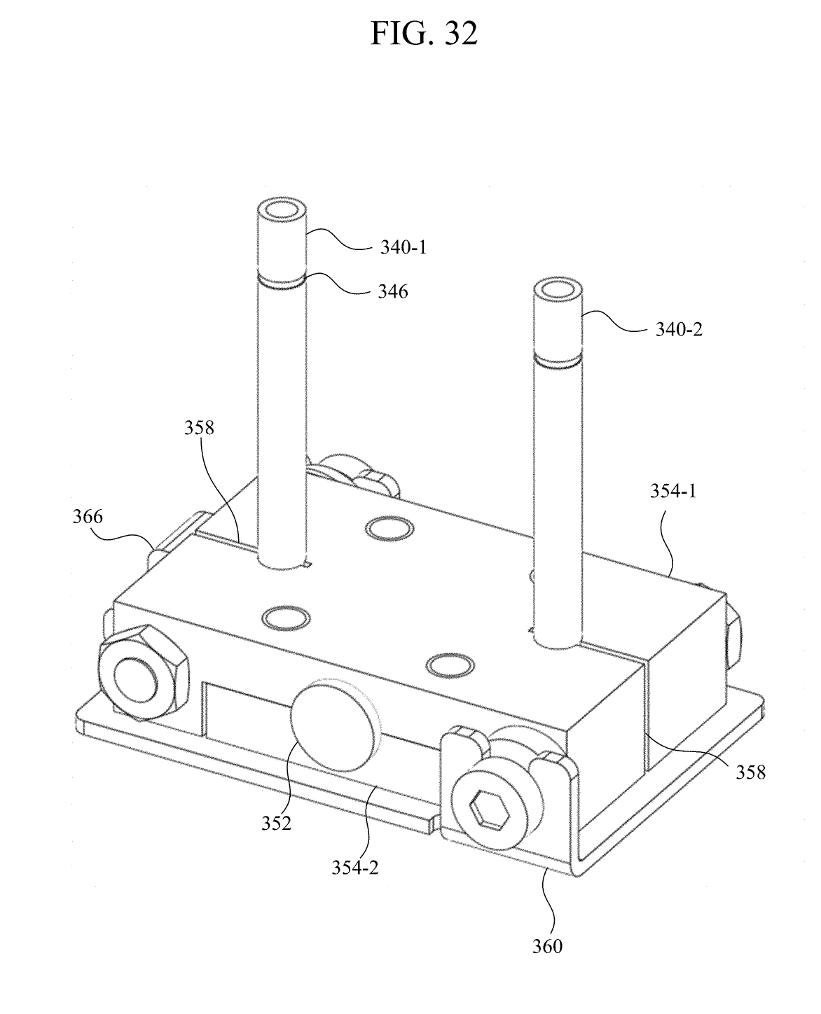

FIG. 33 is a bottom view of a filament extruder, in accordance with an embodiment of the present disclosure;

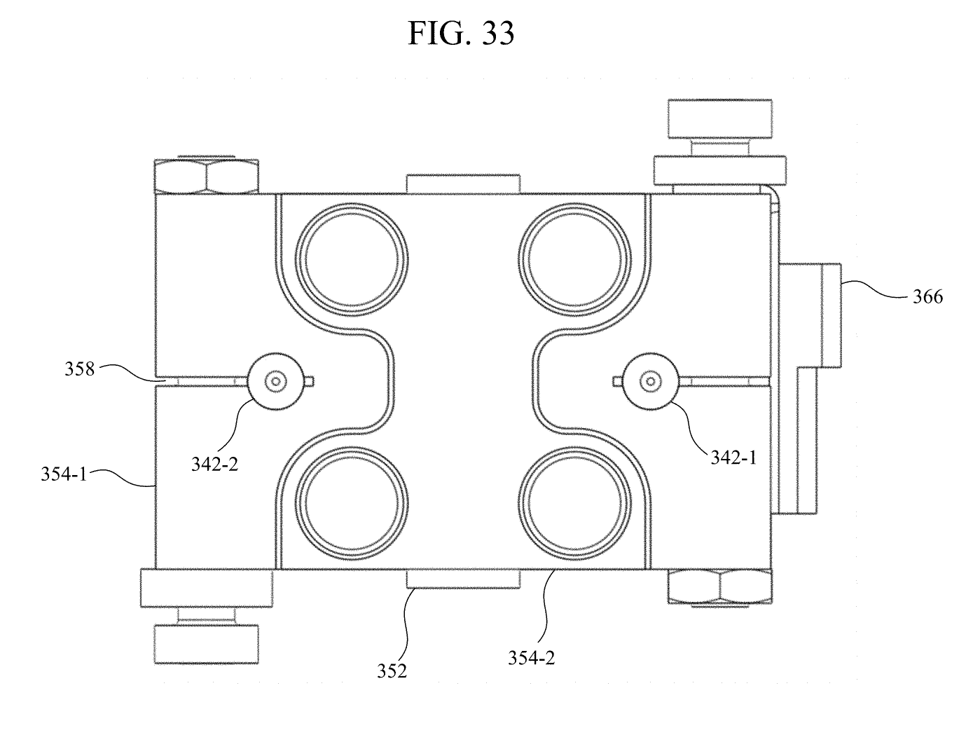

FIG. 34 is a view of a filament extruder, in accordance with an embodiment of the present disclosure;

FIG. 35 is a bottom view a filament extruder, in accordance with an embodiment of the present disclosure;

FIG. 36 is a bottom view of another filament extruder, in accordance with an embodiment of the present disclosure;

FIG. 37 is a bottom view of yet another filament extruder, in accordance with an embodiment of the present disclosure

FIGS. 38A, 38B, 38C, and 38D are views of the multi-compartment capsule according to an embodiment of the present disclosure;

FIGS. 39A, 39B, 39C, 39D, and 39E are views of the multi-compartment capsule according to another embodiment of the present disclosure;

FIGS. 40A, 40B, 40C, and 40D are views of the multi-compartment capsule according to another embodiment of the present disclosure;

FIGS. 41A, 41B, and 41C are views of the multi-compartment capsule according to another embodiment of the present disclosure;

FIGS. 42A, 42B, 42C, 42D, 42E, and 42F are views of the multi-compartment capsule according to another embodiment of the present disclosure;

FIGS. 43A and 43B are views of the multi-compartment capsule according to another embodiment of the present disclosure;

FIGS. 44A and 44B are views of the multi-compartment capsule according to another embodiment of the present disclosure;

FIGS. 45A and 45B are views of the multi-compartment capsule according to another embodiment of the present disclosure;

FIGS. 46A, 42B, and 46C are views of the multi-compartment capsule according to another embodiment of the present disclosure;

FIG. 47 is view of a system for providing a user interface according to an exemplary embodiment of the present disclosure;

FIG. 48 is another view of a system for providing a user interface according to an exemplary embodiment of the present disclosure;

FIG. 49 is a view of the user interface according to an exemplary embodiment of the present disclosure;

FIG. 50 is another view of the user interface according to an exemplary embodiment of the present disclosure;

FIG. 51 is another view of the user interface according to an exemplary embodiment of the present disclosure;

FIG. 52 is another view of the user interface according to an exemplary embodiment of the present disclosure;

FIG. 53 is another view of the user interface according to an exemplary embodiment of the present disclosure; and

FIG. 54 is another view of the user interface according to an exemplary embodiment of the present disclosure.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Reference will now be made in detail to various embodiments of the present invention(s), examples of which are illustrated in the accompanying drawing and described below. While the invention(s) will be described in conjunction with exemplary embodiments, it will be understood that the present description is not intended to limit the invention(s) to those exemplary embodiments. On the contrary, the invention(s) is/are intended to cover not only the exemplary embodiments, but also various alternatives, modifications, equivalents and other embodiments, which may be included within the spirit and scope of the present invention as defined by the appended claims.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject” and “user” are used interchangeably herein.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and or “comprising,” when used in this specification, specify the presence of stated features, integers, steps, operations, elements, and or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

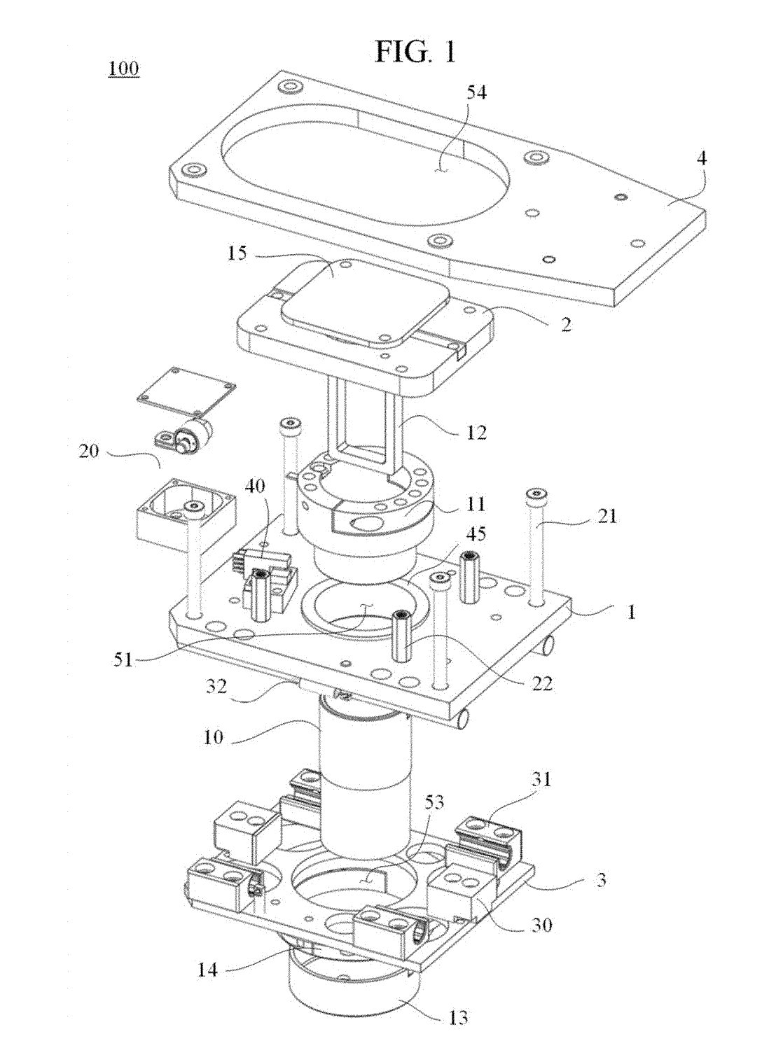

An aspect of the present disclosure is directed to a dispensing system comprising a dispenser 100, a fixing plate 4, a dispensing station 200, and a dial 250. Referring to FIG. 1 to FIG. 7, the dispenser 100, according to an exemplary embodiment of the present disclosure, includes a base 1 formed with a first insertion hole 51 that receives the hopper 10. A vibration device 20 is disposed on a surface of the base 1 and is configured to vibrate the dispenser 100. A plurality of spacers 22 are disposed between an upper base 2 and the base 1. A gate spring 32 is disposed on a bottom surface of the base 1.

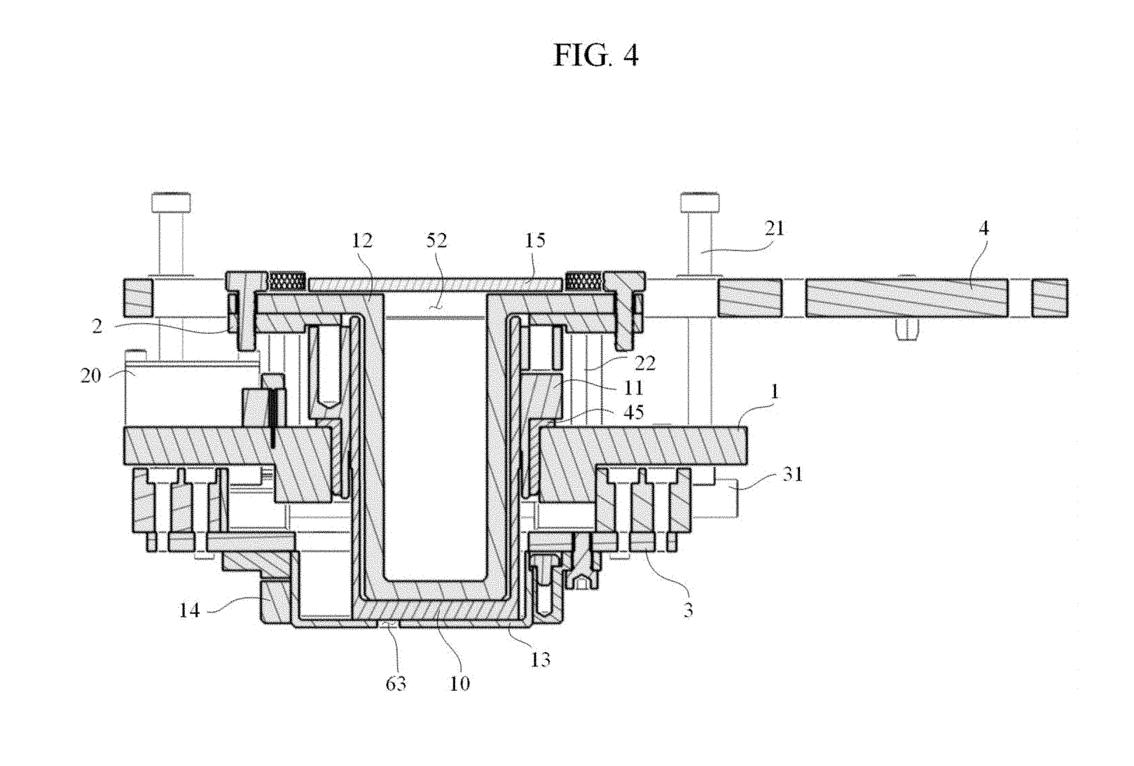

The dispenser 100 also includes an upper base 2 formed with a second insertion hole 52 (FIG. 4) that receives the stirrer 12. The stirrer 12 is fixed to the upper base 2. The stirrer 12 feeds the dispensing material inside the hopper 10 into a plurality (e.g., two or more three or more, ten or more) of metering holes 60. In the present embodiment, the stirrer 12 is formed as a bar shape, however the present disclosure is not limited thereto. For instance, the stirrer 12 could have a chamfer shape to assist the supplying of dispensing material. In some embodiments, the stirrer 12 is formed in an auger or plate shape, and in another embodiment the stirrer 12 is formed as a bent tube.

The dispenser 100 also includes a lower base 3 formed with a third insertion hole 53 that accommodates a gate 13. A second end of the gate spring 32 is disposed on an upper surface of the lower base 3. A pair of linear guides 31 couple the lower base 3 to the base 1.

In some embodiments, the dispenser 100 further includes a hopper hub 11, a gate hub 14, a lid 15, a plurality of spring-dampers 21, a transfer block 30, a detector 40, and a bearing 45 as further described below.

The base 1 has a plate shape and is formed with the first insertion hole 51 at the center thereof. As described above, the first insertion hole 51 into or from which the hopper 10 can be inserted or withdrawn, is formed through the broad surface of the base 1. The vibration device 20 is disposed on the upper surface of the base 1, but is not limited thereto. For instance, the vibration device 20 may be disposed on the bottom surface of the base 1. The size and actuation pattern of the vibration device 20 can be varied to create a desired vibration frequency and amplitude according to a design by one skilled in the art.

The upper base 2 has a similar plate shape as the base 1, and is formed with the second insertion hole 52 at the center thereof. As described above, the second insertion hole 52, into which the stirrer 12 can be inserted and fixed, is formed through the broad surface of the upper base 2. In some embodiments, a recessed groove is formed on the upper surface of the upper base 2 from which the stirrer 12 is accommodated. The stirrer 12 is inserted through the second insertion hole 52 and is received by the hopper 10.

The illustrated embodiment is configured with the lid 15 disposed on the upper surface of the upper base 2. This isolates the contents of the hopper 10 from an external environment. In the illustrated embodiment, the lid 15 is formed from a transparent material so that the contents of the hopper 10 may be examined during operation. However, the present disclosure is not limited thereto. For instance, in some embodiments the lid 15 is formed of a different material such as metal or plastic, or is entirely omitted. Also, in the illustrated embodiment, the lid 15 is coupled to the upper surface using screws, however the lid 15 is coupled using magnets, a hinge, or a similar mechanism in other embodiments.

The spacers 22 are disposed on the bottom surface of the upper base 2 and the upper surface of the base 1. A gap is formed between the hopper 10 and the stirrer 12. The height of the spacers 22 determines the height of the gap between the hopper 10 and the stirrer 12. The height of the gap is configured so that the clearance is sufficiently large to prevent galling and sufficiently small to prevent the dispensing material from entering the gap. In some embodiments, the height of the gap ranges from 0 mm to 3 mm. In the illustrated embodiment, the spacers 22 are configured as standoffs, however the present disclosure is not limited thereto.

The lower base 3 has a similar plate shape as the base 1 and is formed with the third insertion hole 53 at the center thereof. As described above, the third insertion hole 53 into or from which the hopper 10 can be inserted or withdrawn, is formed through the broad surface of the lower base 3. The aperture of the third insertion hole 53 is larger than a diameter of the hopper 10 so that the lower base 3 is slideable about the hopper 10. In some embodiments, the aperture of the third insertion hole 53 ranges from 15 mm to 230 mm. The gate 13 is disposed on the bottom surface of the lower base 3 and is mounted to the lower base 3 using the gate hub 14. The gate 13 is disposed such that there is a gap formed between the hopper 10 and the gate 13. In some embodiments, the gap between the hopper 10 and the gate 13 ranges from 0 mm to 3 mm. In some embodiments, the hopper 10 has a diameter which ranges from 10 mm to 200 mm. The height of the hopper 10 determines the amount of the gap between the hopper 10 and the gate 13. The amount of the gap (e.g., height, width, etc.) is configured so that the clearance is sufficiently large to prevent galling and sufficiently small to prevent the dispensing material from entering the gap.

The lower base 3 also comprises a second end of a gate spring 32 disposed on the upper surface of the lower base 3. The first end of the gate spring 32 is disposed on the bottom surface of the base 1. The gate spring 32 provides a restoring force in parallel with the sliding motion of the lower base 3.

The lower base 3 is coupled to the base 1 using a pair of linear guides 31. The linear guides 31 are configured to restrict the motion of the lower base 3 when switching between ON and OFF states.

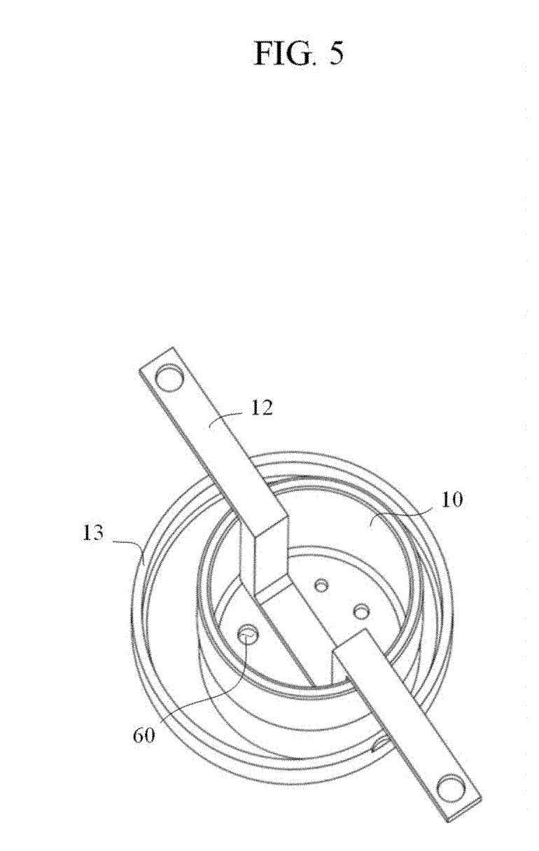

Referring to FIG. 5, the hopper 10 is formed with one or more metering holes 60 on the bottom surface thereof. The volume defined by the one of more metering holes 60 defines a predetermined volume of material to be dispensed. In the present embodiment, an array of metering holes 60 are disposed on the bottom surface of the hopper 10, but the present disclosure is not limited thereto. For instance, the metering holes 60 may only be a singular metering hole 60, or be formed in a variety of shapes such as a rectangular prism or a frustum of a right circular cone. As mentioned above, the hopper 10 is inserted through the first, second, and third insertion holes 51, 52, and 53, and is sandwiched between the gate 13 and the hopper hub 11.

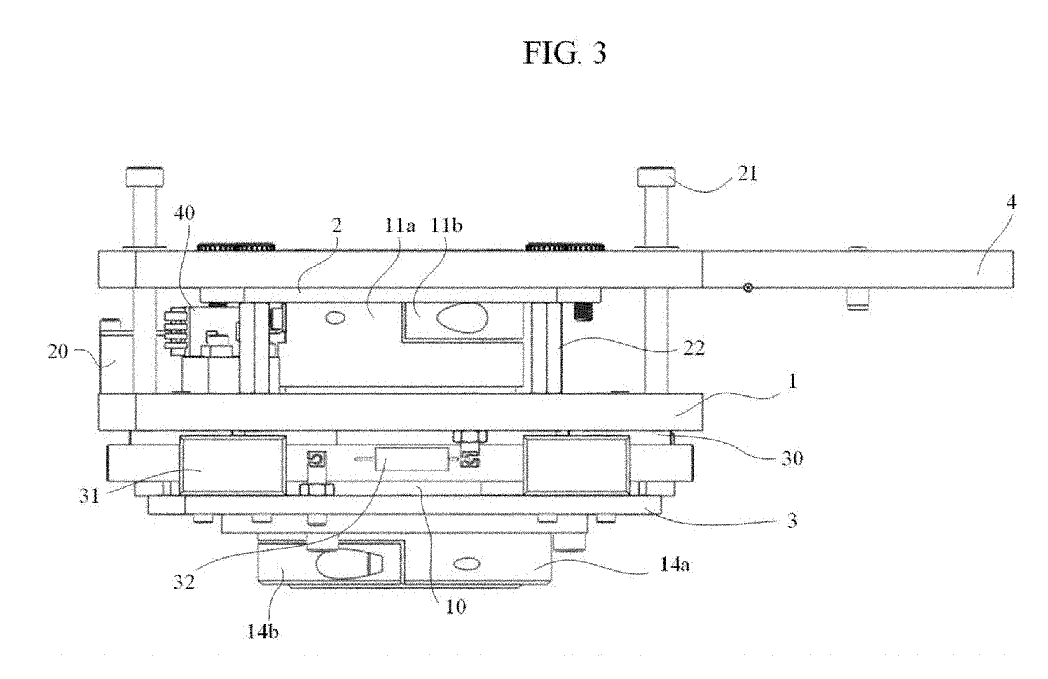

The hopper hub 11 is disposed between the hopper 10 and the bearing 45, and is configured to fix an upper portion of the hopper 10 as well as transfer the energy of the drive wheel 230 to the hopper 10. As shown in FIG. 3, the hopper hub 11 is formed from two coupled hopper hub portions 11a and 11b, and has a shape in which the upper portion has a diameter larger than the first insertion hole 51, and the lower potion has a diameter slightly less than the first insertion hole 51. In some embodiments, the first insertion hole 51 has a diameter that ranges from 10 mm to 200 mm. In some embodiments, the hopper hub 11 sits on the first insertion hole 51. In some embodiments, the hopper hub portions 11a and 11b are separated to allow the hopper 10 to be withdrawn from the first insertion hole 51 for cleaning and maintenance.

The bearing 45 is disposed between the hopper hub 11 and the base 1, and is configured to reduce friction between the two members. In the present embodiment, the bearing 45 is disposed between the hopper hub 11 and the base 1, however the present disclosure is not limited thereto. For instance, in some embodiments, the bearing 45 is omitted and the hopper hub 11 and the base 1 are in close contact.

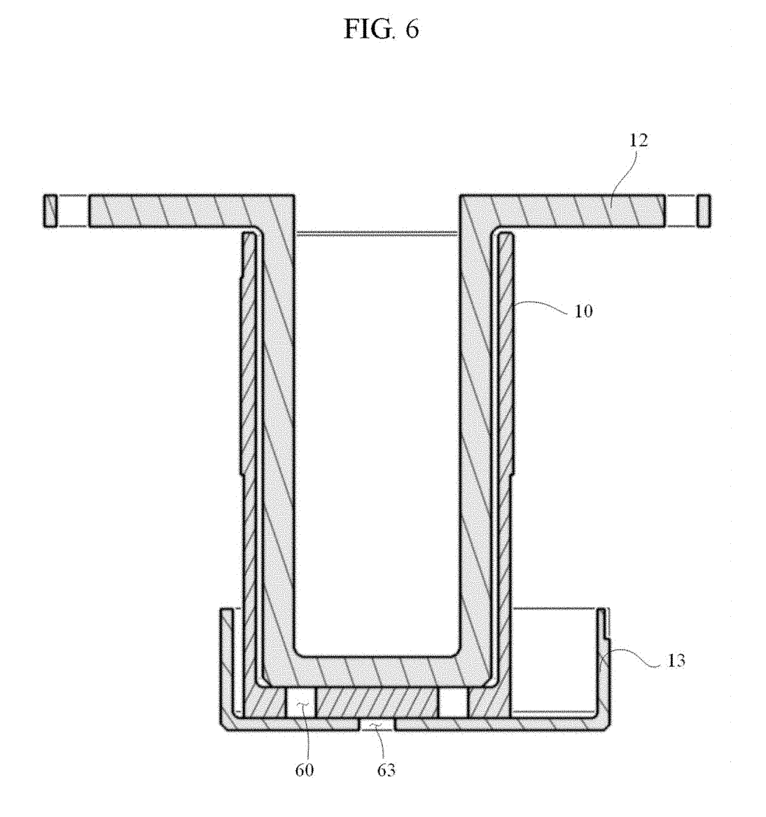

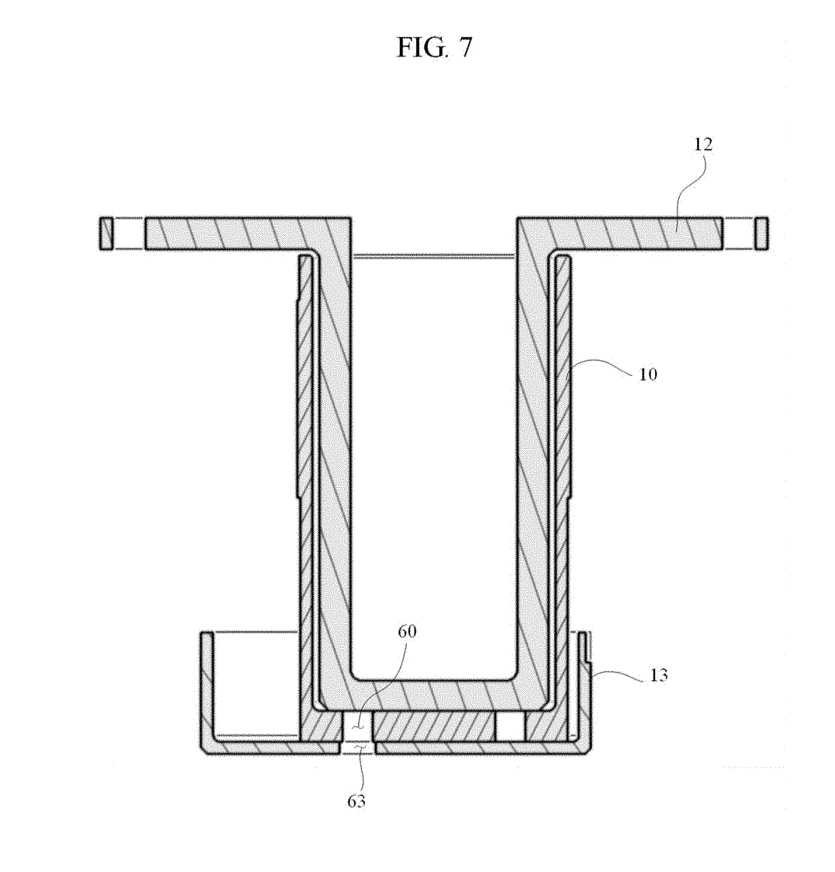

Referring to FIG. 6 and FIG. 7, the gate 13 is formed with a dispensing hole 63 on the bottom surface thereof and an open top end. The top end of the gate 13 surrounds and encloses the hopper 10 and a diameter of the gate 13 is equivalent to the diameter of the third insertion hole 53. The dispensing hole 63 is formed so that the dispensing hole 63 is aligned with a selected metering hole 60 when in the ON position, and misaligned with all metering holes 60 when in the OFF position. The diameter of the gate 13 is sufficiently large that the gate 13 may slide beneath the hopper 10 to allow the above ON or OFF configurations.

The gate hub 14 is disposed between the gate 13 and the lower base 3, and fixes the gate 13 to the lower base 3. In the present embodiment, the gate hub 14 is formed from two coupled gate hub portions 14a and 14b. Gate hub portions 14a of the gate hub 14 are mounted to the lower base 3. Gate hub portion 14b is coupled to the gate hub portion 14a. The gate hub portion 14b is removable. In this way, the hopper 10 and the gate 13 are withdrawn for cleaning and maintenance in some embodiments.

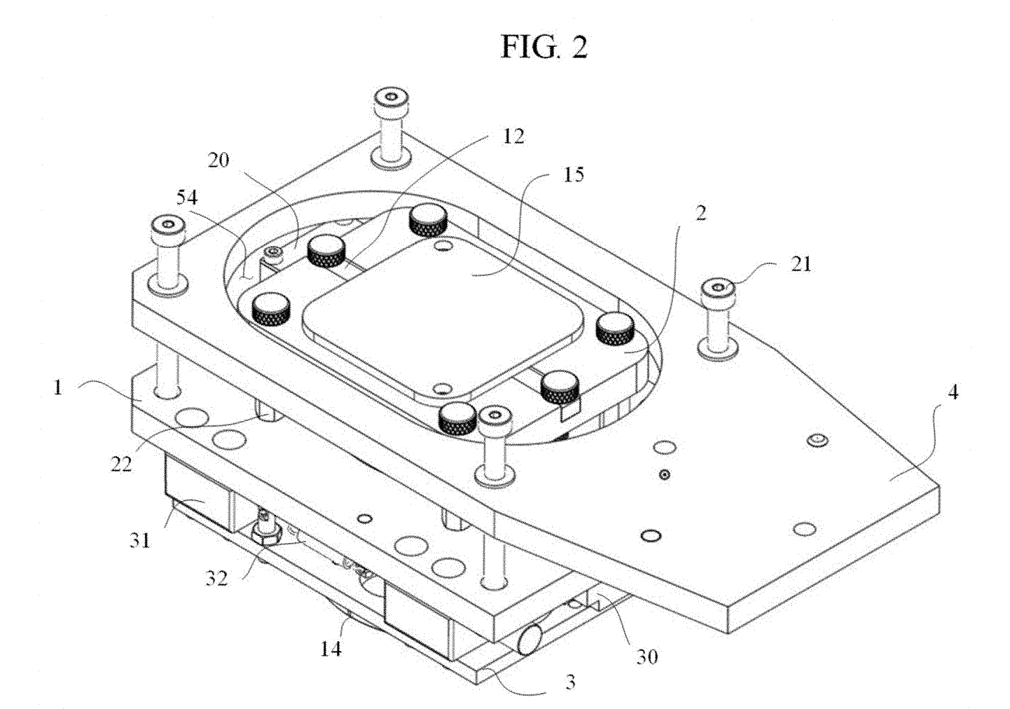

As shown in FIG. 2, the fixing plate 4 is formed with a fourth insertion hole 54 in the center thereof and fixes the dispenser 100 to the dispensing station 200. The fourth insertion hole 54 is formed in such a way that the aperture of the fourth insertion hole 54 is sufficiently large to accommodate the upper base 2 and prevent the upper base 2 and the fixing plate 4 from abutting during operation. The fixing plate 4 is coupled to the dispenser 100 using a plurality of spring-dampers 21. The spring-dampers 21 have (e.g., comprises) a first end disposed on the base 1 and a second end disposed through the fixing plate 4. The spring-dampers 21 isolate the vibration of the vibration device 20 so that only the dispenser 100 is agitated and oscillates as a whole assembly. In some embodiments, the size, elasticity, and damping force of the spring-dampers 21 varies depending on the design requirements. The present configuration is advantageous compared to the conventional dispensers, as the present disclosure prevents micro-impacts between members that vibrate independently, thereby improving the lifespan of the apparatus and reducing the required maintenance.

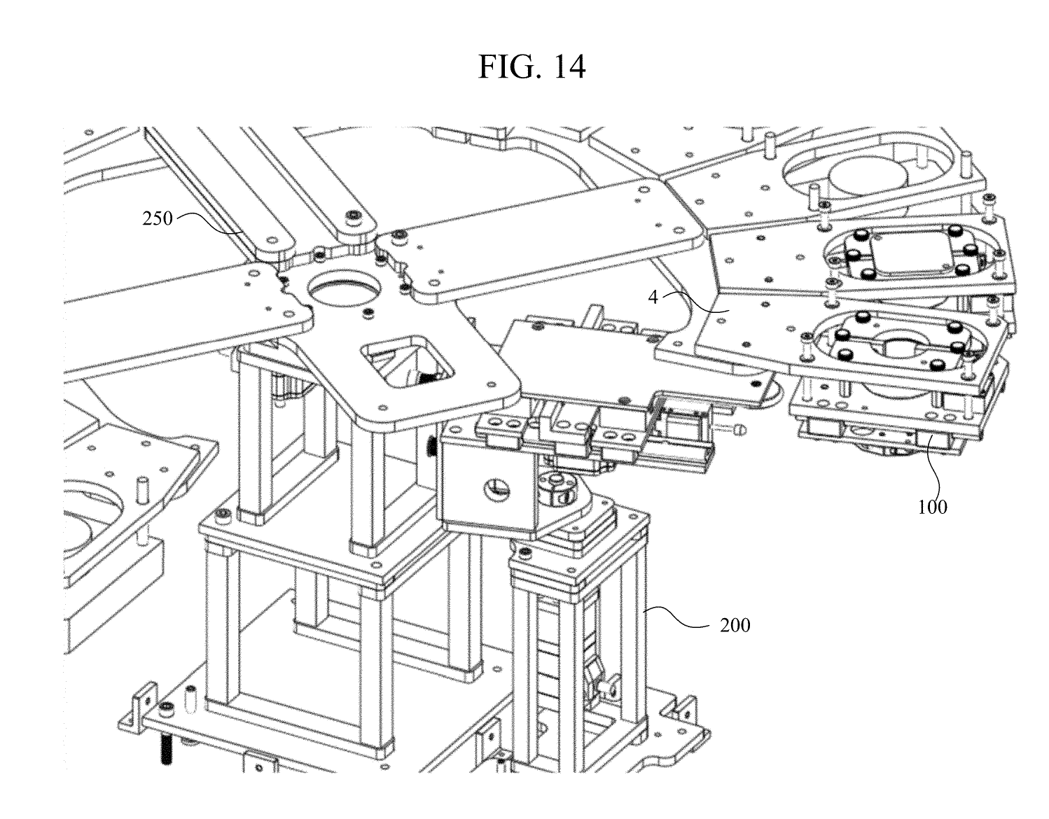

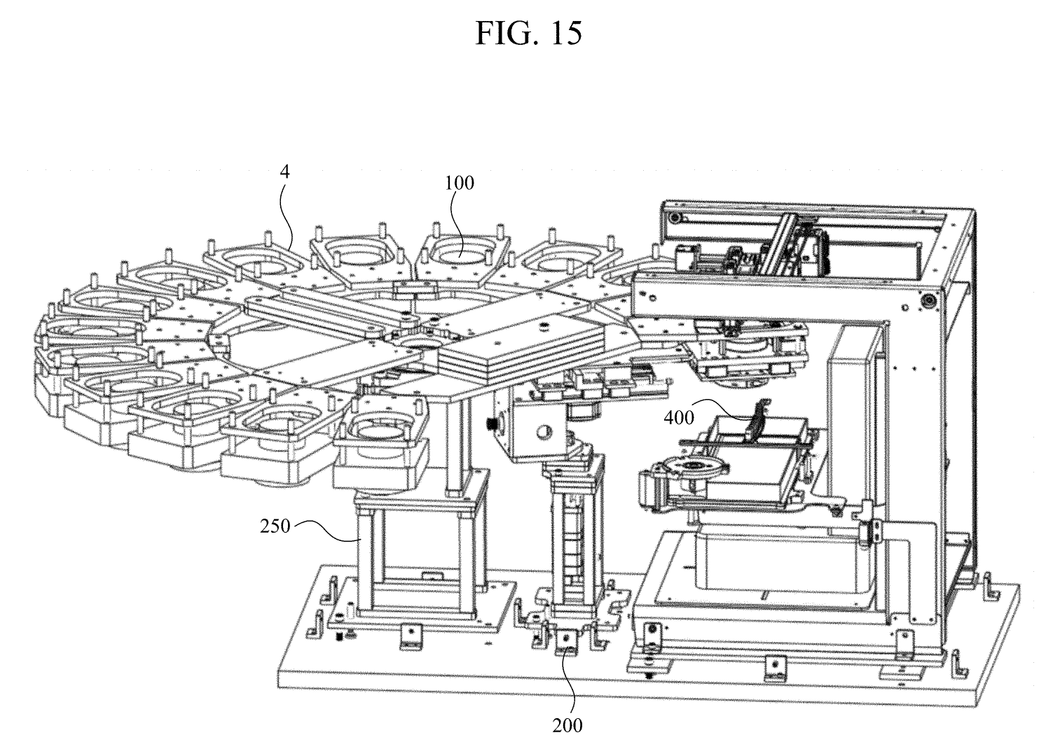

Referring to FIG. 14 and FIG. 15 in some embodiments dial 250 is fixed to an end of the fixing plate 4 instead of the dispensing station 200. The dial 250 is configured to accommodate a plurality of the dispensers 100. The dial 250 is configured as a library of dispensers 100 (e.g., two dispensers, three four dispensers, four dispenser or more, 10 or more dispensers, or 20 or more dispensers), where a selected dispenser 100 can be oriented to the dispensing station 200. In some embodiments, the dial 250 comprises a gantry or robotic arm.

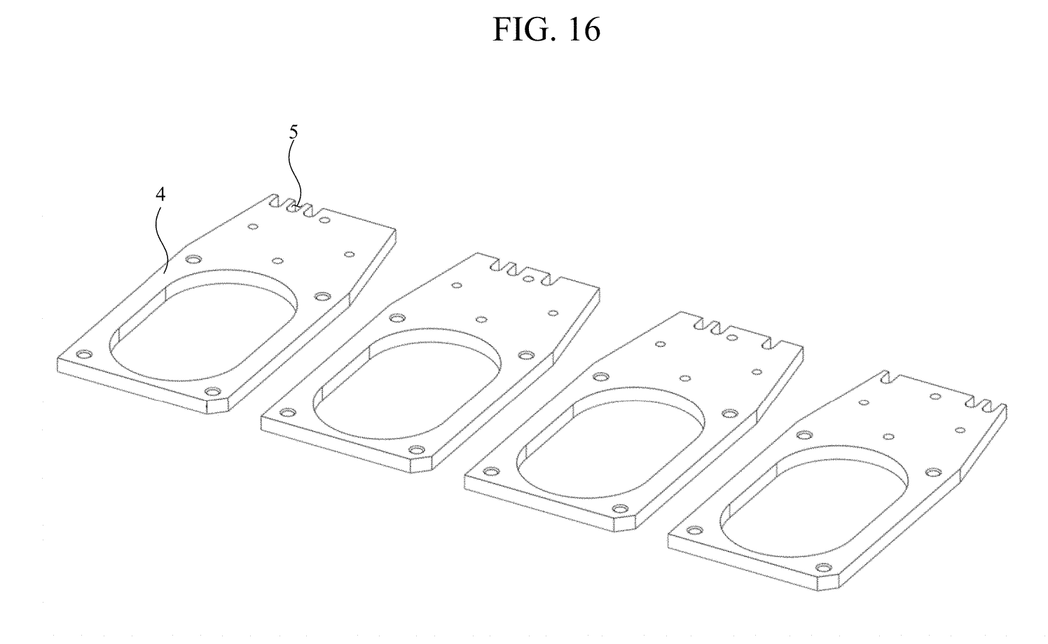

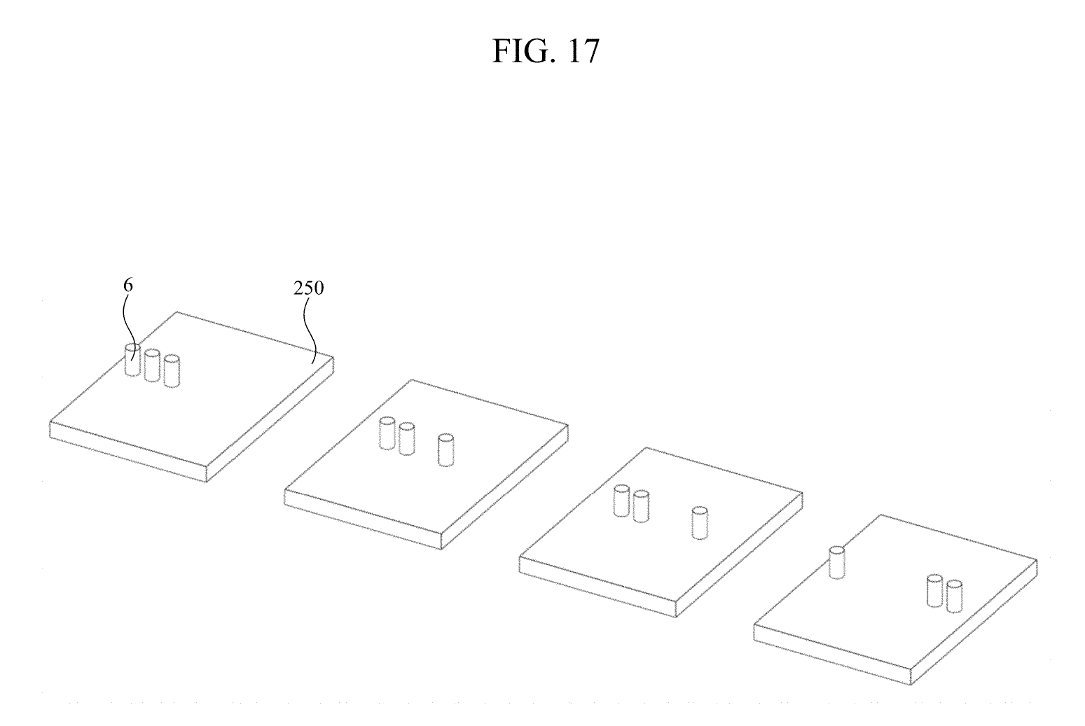

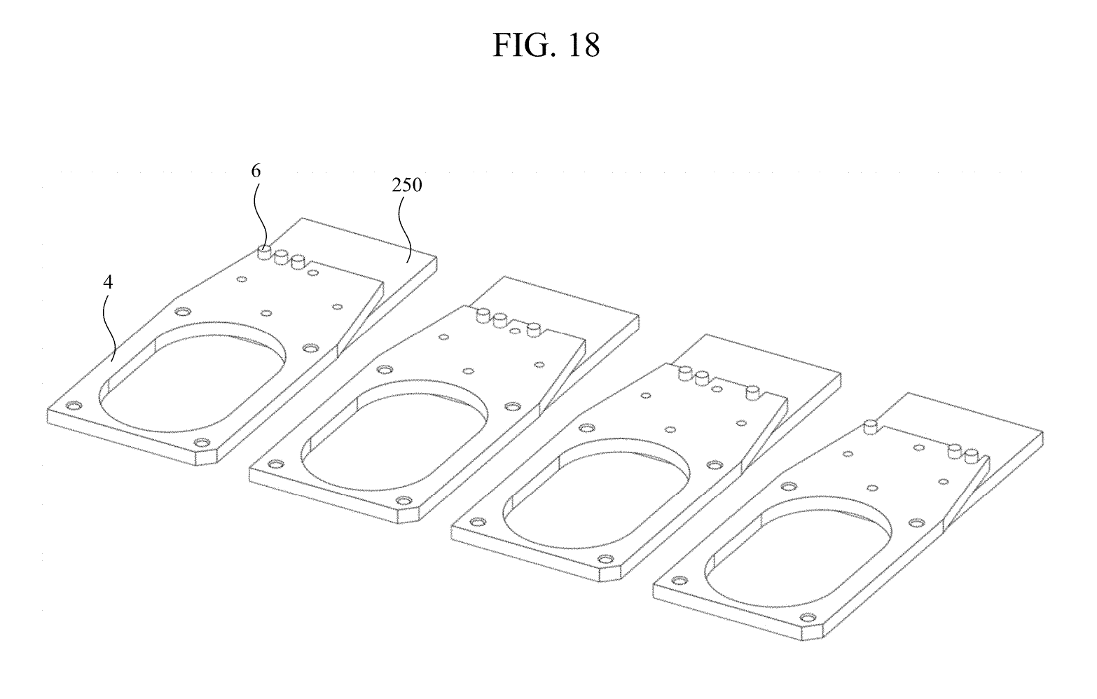

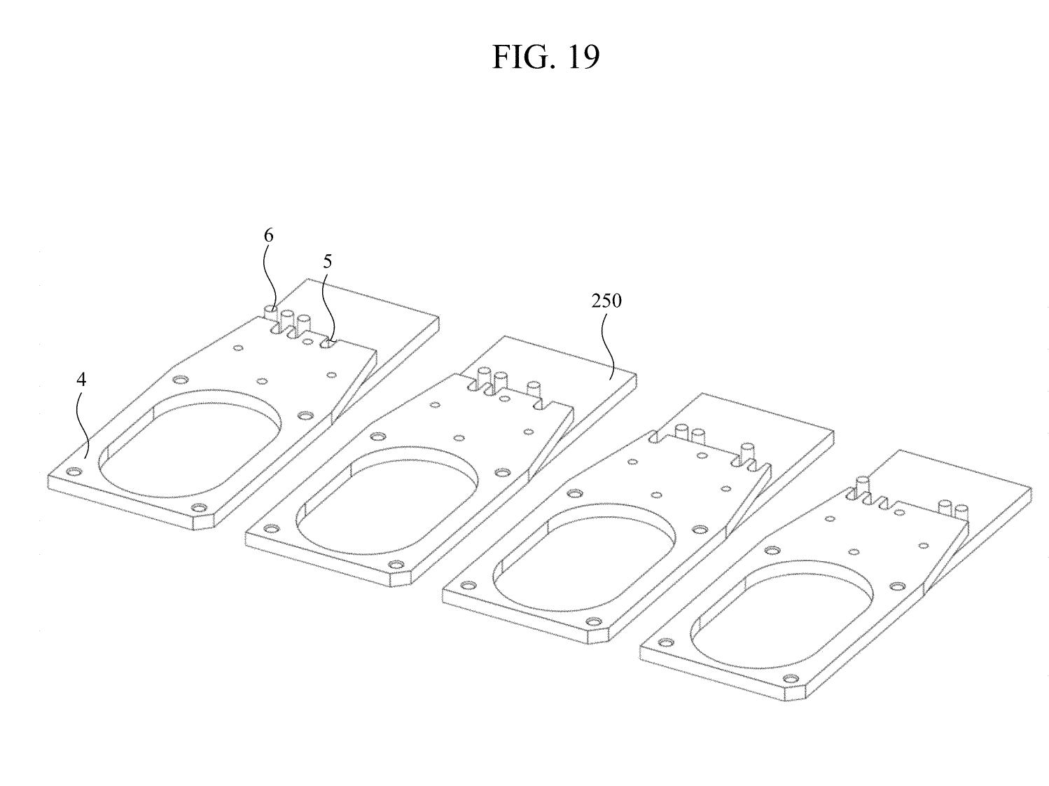

Referring to FIG. 16 to FIG. 19, the fixing plate 4 and the dispenser 250 comprise a peg 6 and a notch 5 identification (ID) system. A plurality of dispensers 100 are fixed to the dial 250. Each fixing plate 4 comprises a unique array of the notches 5 disposed on an edge thereof. Each arm of the dial 250 comprises a matching array of the pegs 6 formed in such a way that each dispenser 100 can only be fixed to the matching arm of the dial 250. As shown in FIG. 19, when the notch 5 and peg 6 are not aligned, the dispenser 100 cannot be fixed to the dial 250 thereby ensuring the correct dispenser is fixed to the correct arm of the dial 250. The present configuration ensures that a computer control system (not shown) always knows which of the dispensers 100 is located and which dispenser 100 should be activated.

In some embodiments, a detector 40 is disposed on the dispenser 100, and is configured to communicate with a central controller (not shown). In some such embodiments, the detector 40 communicates the position, velocity, or acceleration of the hopper 11, and or the amplitude or frequency of the vibration device 20.

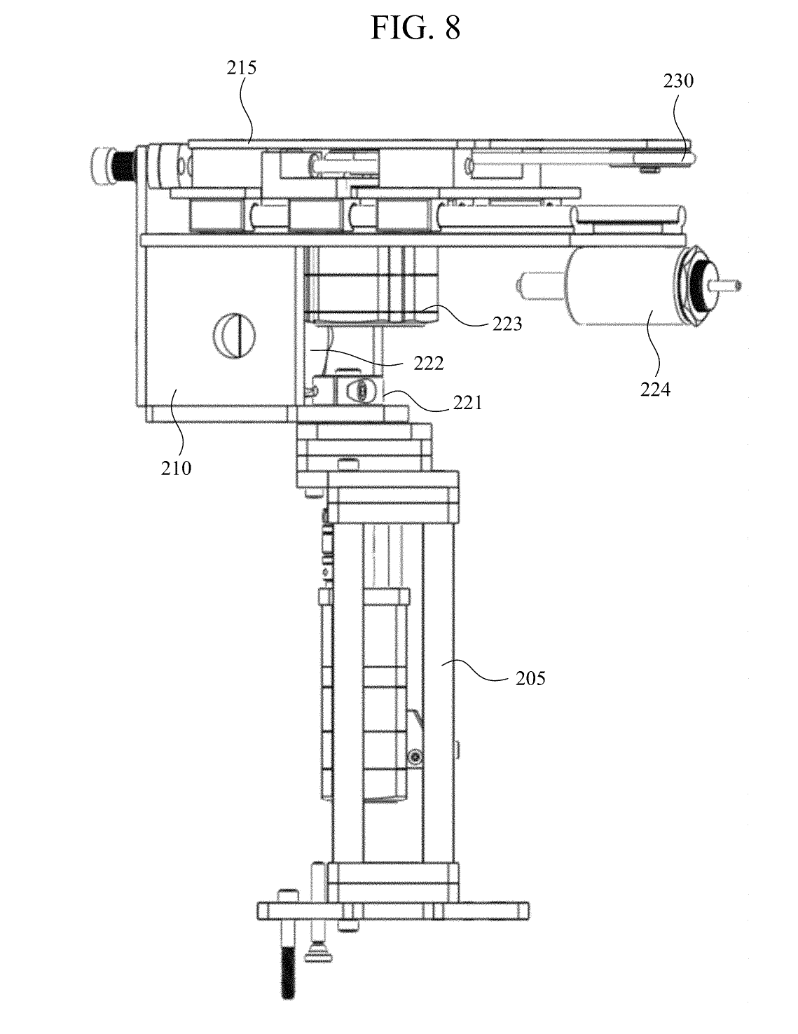

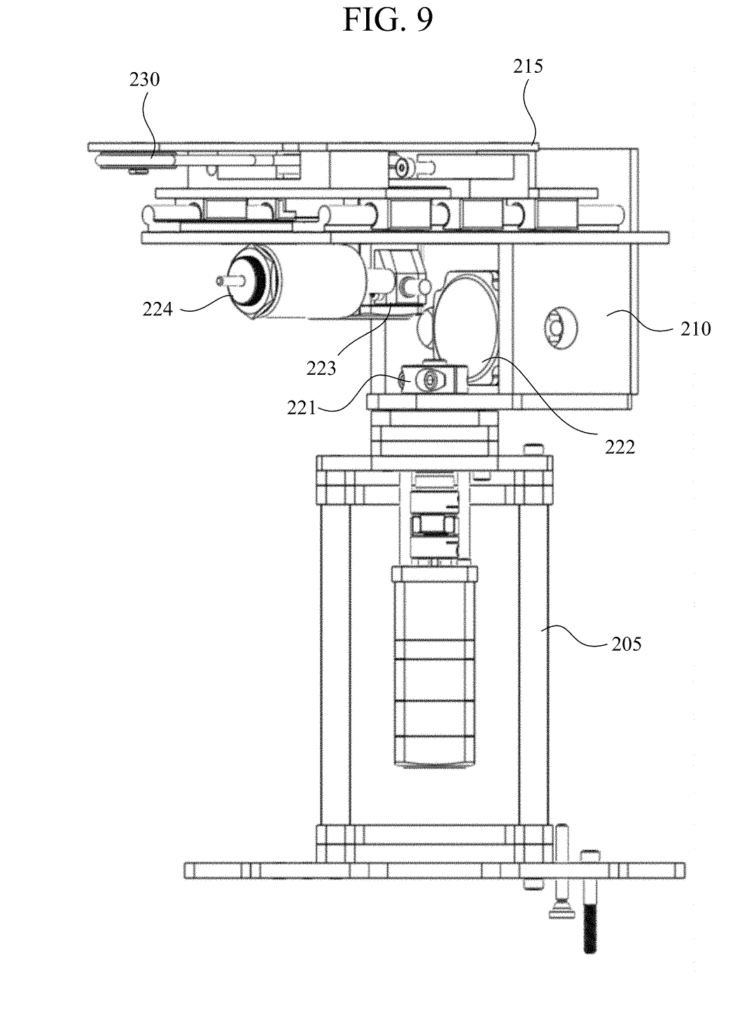

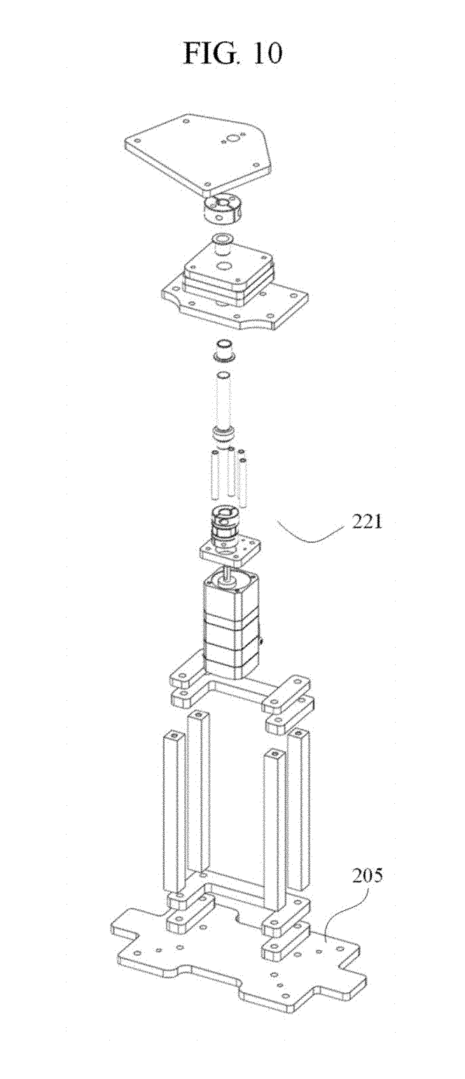

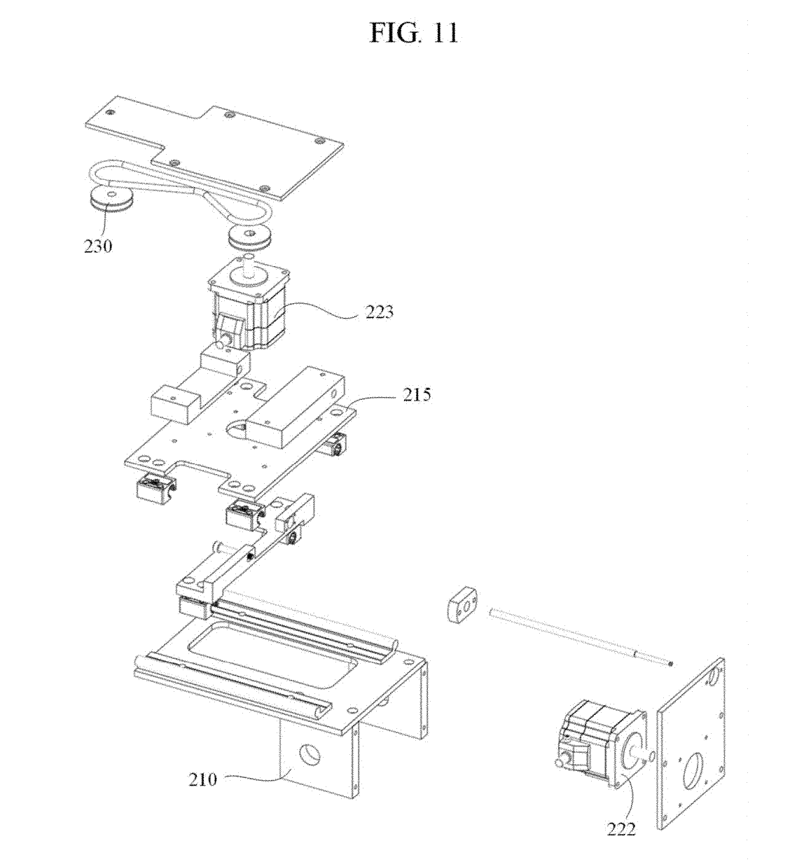

Referring to FIG. 8 to FIG. 11, the dispensing station 200 of one aspect of the present disclosure comprises a stationary base 205 configured to be fixed at the center of rotation of the system and act as a central anchor to the dispensing station 200. The station further comprises an upper base 215 housing the second, third, and fourth actuators 222, 223, and 224. The station further comprises a platform 210 that distances the upper base 215 and the stationary base 205. The station further comprises a first actuator 221 that rotates the upper base 215 about a horizontal plane, a second actuator 222 that engages a drive wheel 230 with the hopper hub 11, a third actuator 223 that rotates the drive wheel 230, and a fourth actuator 224 that engages a transfer block 30.

The stationary base 205 mounts the dispensing system to a desired external mounting fixture. The lower end of the stationary base 205 is fixed and the upper end of the stationary base 205 is disposed with the first actuator 221. In the present embodiment, the stationary base 205 is formed from a plurality of individual plates and bars. The total height and modularity of the stationary base 205 is adjusted, serving as a counterweight, however the present disclosure is not limited thereto. For instance, in some embodiments the base 205 is formed integrally. The first actuator 221 is configured to couple the stationary base 205 with the platform 210 and to rotate and orient the platform 210 and the upper base 215. In some embodiments, the first actuator 221 is coupled to the dial (not shown) and rotates and orients a dispenser 100 from a plurality of dispensers 100 to the dispensing station 200.

As described above, the platform 210 is coupled to the stationary base 205 using the first actuator 221. The upper end of the platform 210 is coupled to the upper base 215 using the second actuator 222. The second actuator 222 is configured to slide the upper base 215 along the platform 210 wherein the drive wheel 230 will engage and disengage with the hopper hub 11 according to the sliding motion of the upper base 215.

The upper base 215 is disposed above the platform 210 and houses the drive wheel 230 and the third and fourth actuators 223 and 224. The drive wheel 230 is transfers rotational energy from the third actuator 223 to the hopper hub 11. In the present embodiment, the drive wheel 230 is a drive belt, but is not limited thereto. The third actuator 223 drives the drive wheel 230. The fourth actuator 224 is disposed below the third actuator 223 and engages the transfer block 30 of the dispenser 100. The fourth actuator 224 is responsible for switching the gate 13 to or from the ON or OFF positions. In the present embodiment, the transfer block 30 is disposed on the lower base 3 to assist the fourth actuator 224 in engaging the assembly. However, the present disclosure is not limited thereto. For instance, in alternative embodiments, the fourth actuator 224 engages the lower base 3 or engages the gate 13 directly, thereby omitting the transfer block 30.

In some embodiments, the dispensing station 200 comprise a central controller (not shown) that controls the vibration device 20, the detector 40, the dial (not shown), and the actuators 221, 222, 223, and 224. The central controller may be physical hardware (e.g., one or more microprocessors), software (e.g., drivers) on an external computer, or combinations thereof. In some embodiments, the controller may be a part of a separate computing system.

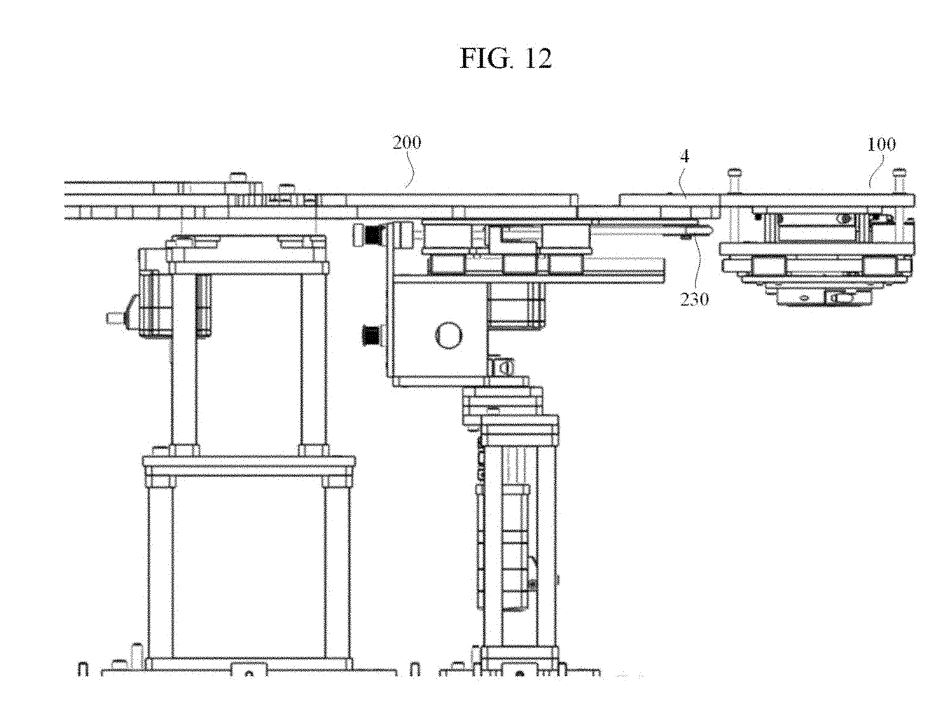

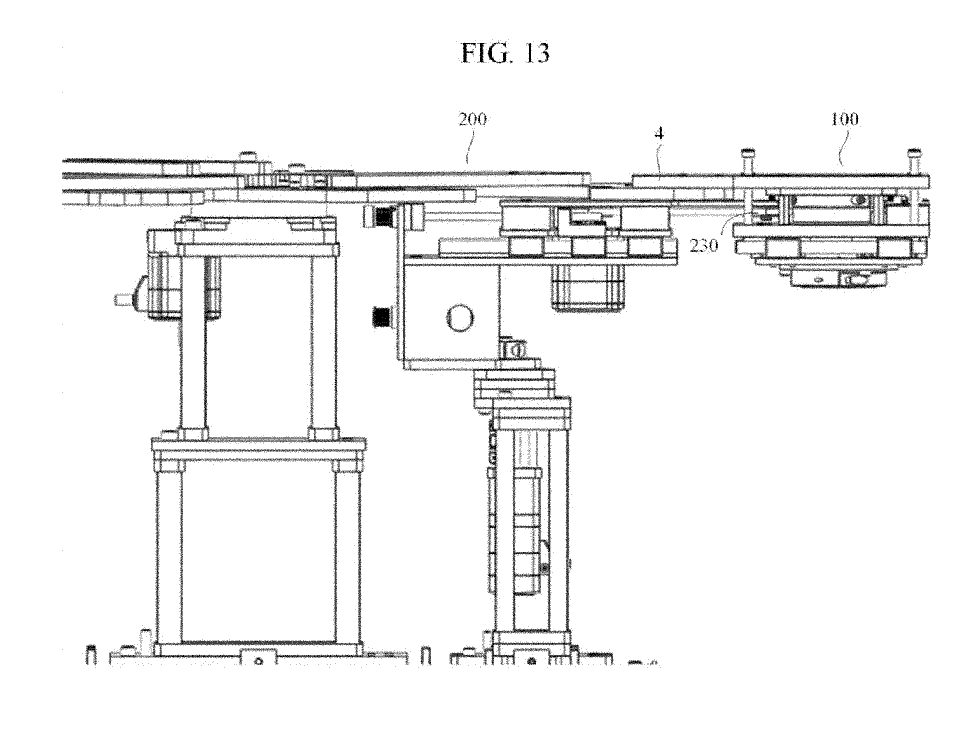

Referring to FIG. 6, FIG. 7, FIG. 12 and FIG. 13, the operation of the dispenser 100 and dispensing station 200 are exemplified. In the OFF position, the second actuator 222 slides the upper base 215 towards the dispenser 100. In the extended ON position, the drive wheel 230 is engaged with the hopper hub 11. As the third actuator 223 drives the drive wheel 230, the rotational energy is transferred to the hopper hub 11 and the hopper 10, thereby rotating the hopper 10 about the stirrer 12. As the hopper 10 rotates, the dispensing material is supplied to the metering holes 60 using the stirrer 12. Once a selected metering hole 60 has been filled, the hopper 10 rotates wherein the selected metering hole 60 is covered by the stirrer 12 to prevent additional dispensing material from entering the selected metering hole 60. The fourth actuator 224 then engages with the transfer block 30 so that the lower block 3 and its constituent components, including the gate 13, slide with the transfer block 30. The lower base 3 slides to a position in which the dispensing hole 63 and the selected metering hole 60 are aligned in the ON position. The vibration device 20 is activated to promote the flow of dispensing material, and the dispensing material is free to flow from the metering hole 60 through the dispensing hole 63 and onto a target surface. As the fourth actuator disengages the transfer block 30 the restoring force of the gate spring 32 forces the lower base 3 to its initial position. In the case that a second, or multiple, dispensing materials are required, the first actuator 221 orients a second dispenser 100 of the dial 250 to the dispensing station 200, and the above process is repeated.

In some embodiments, material is supplied to a single metering hole 60 and then released. In another embodiment, material is supplied into a sequence of metering holes 60 and then released in a predetermined sequential order. In a further embodiment, material is supplied into a sequence of metering holes 60 in a sequence of dispensers 100, and then released in a predetermined sequential order.

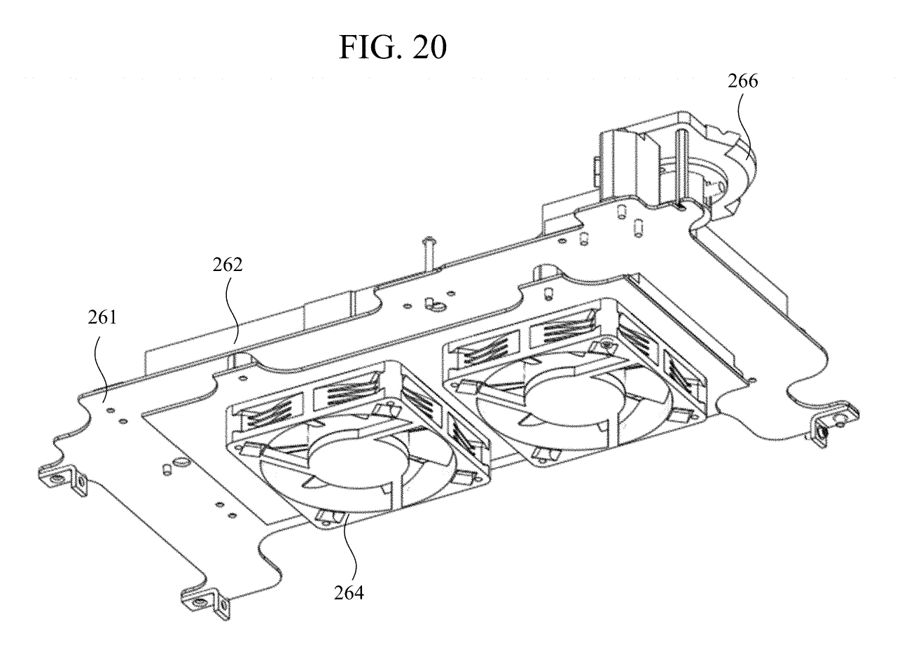

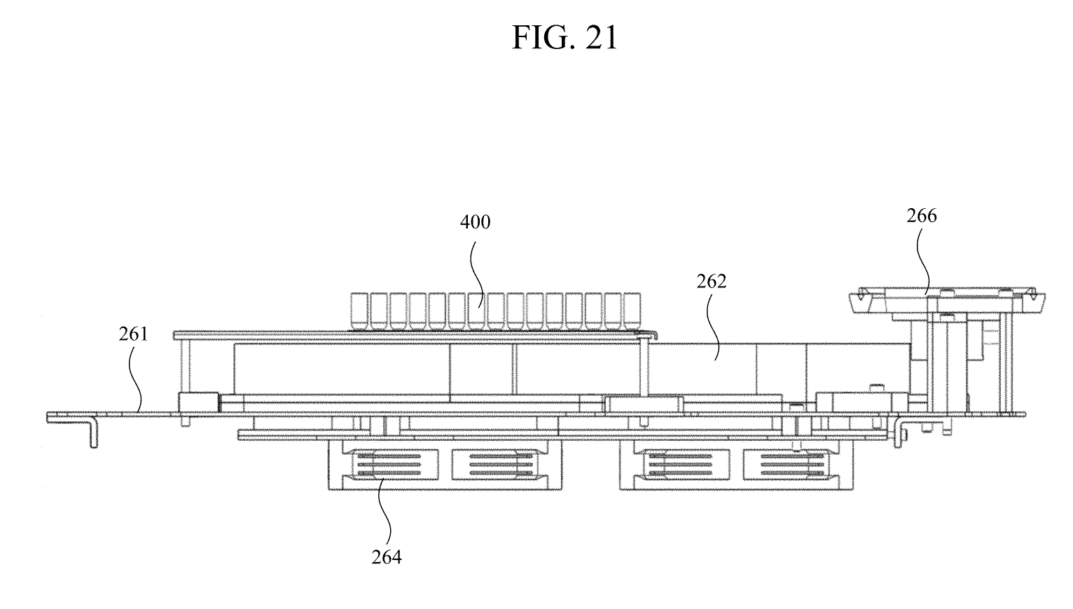

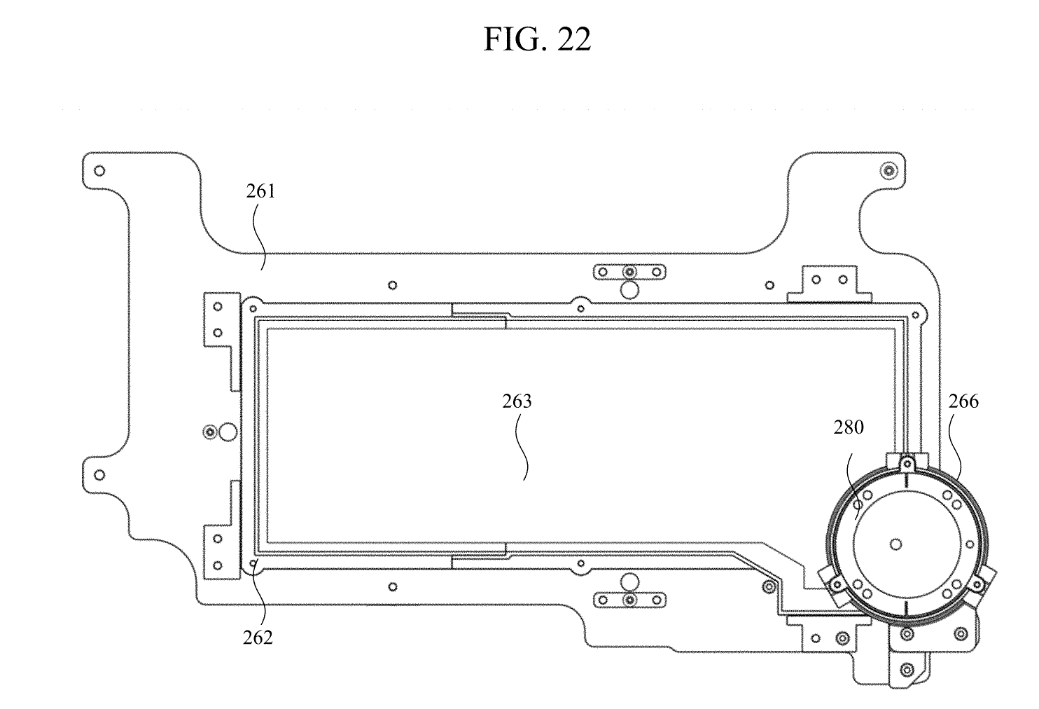

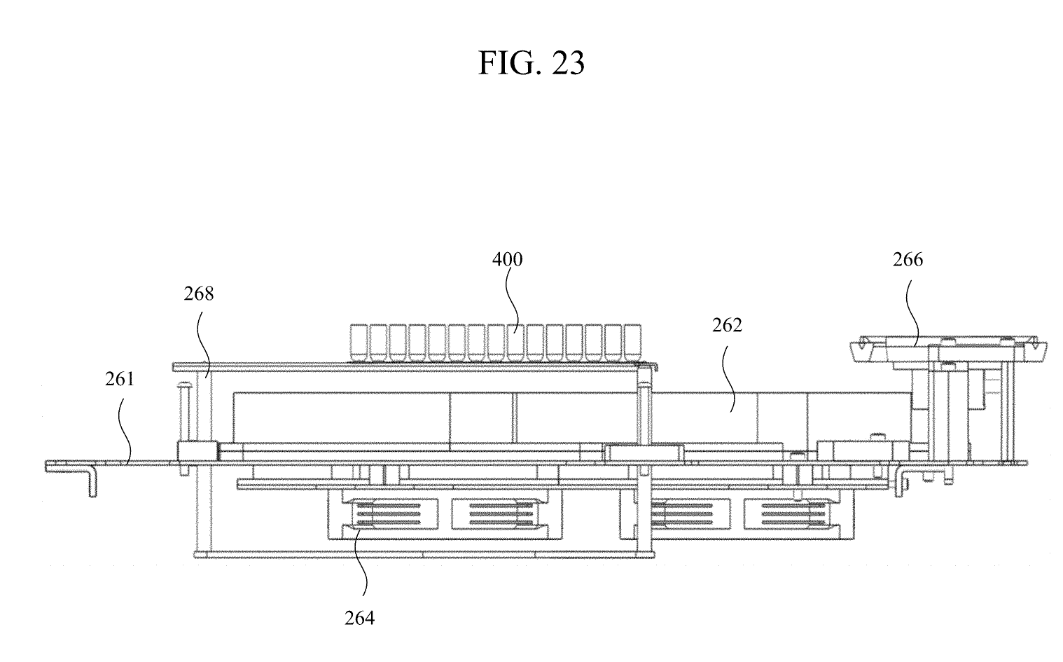

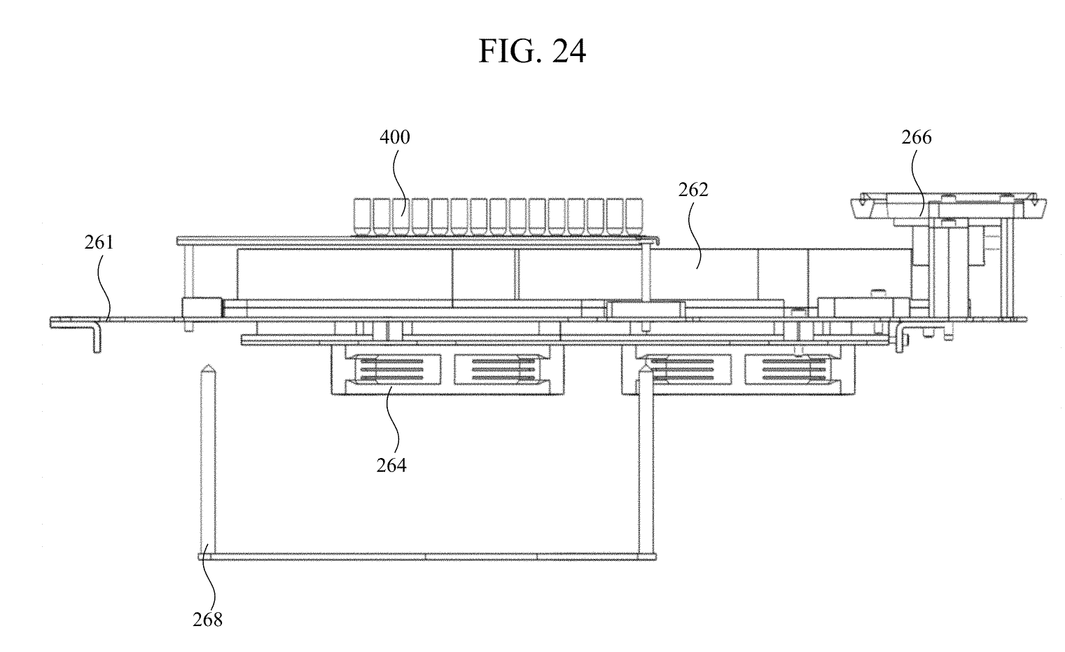

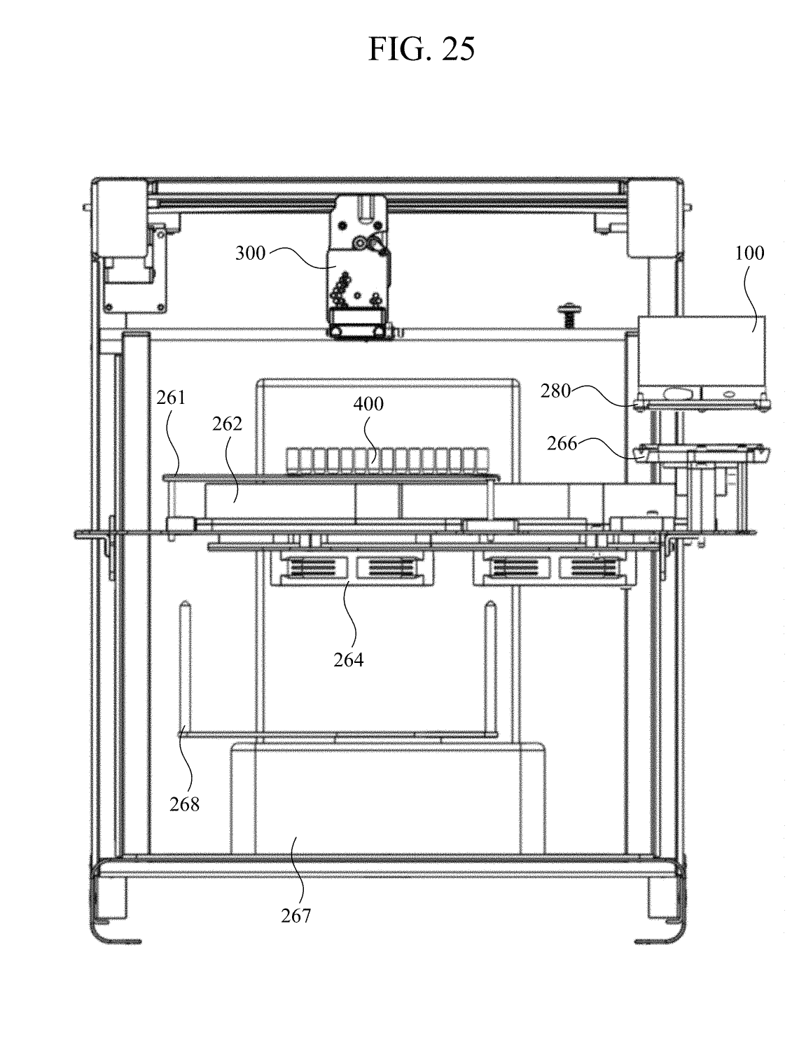

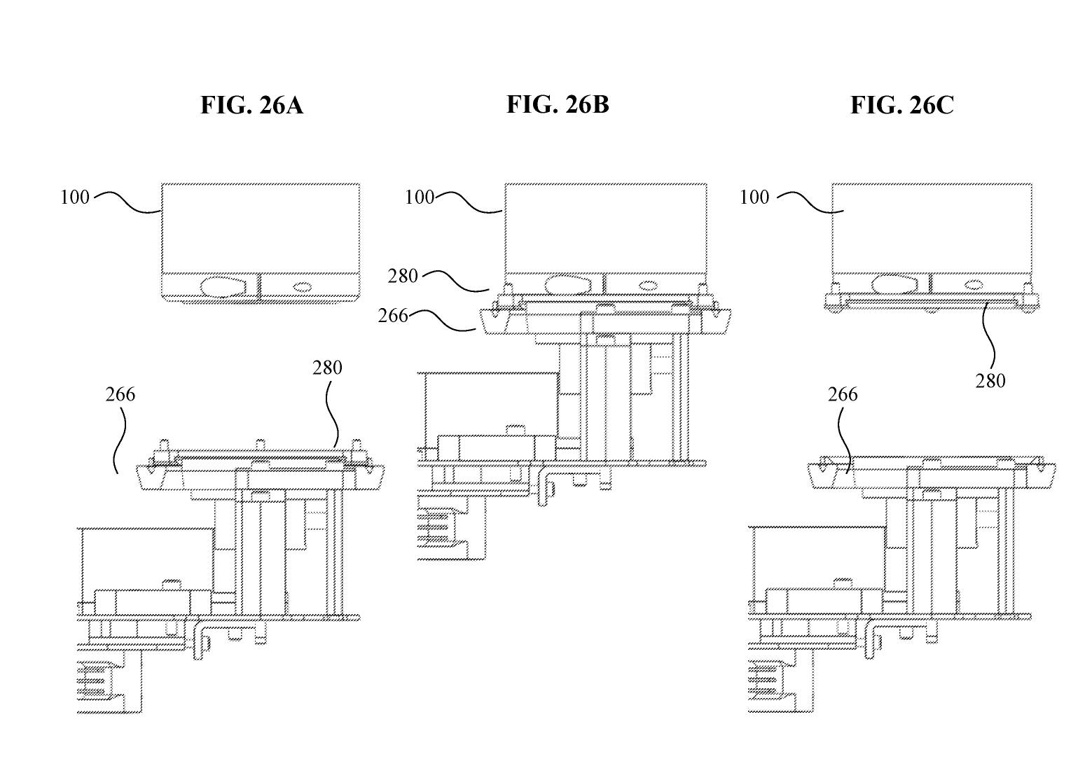

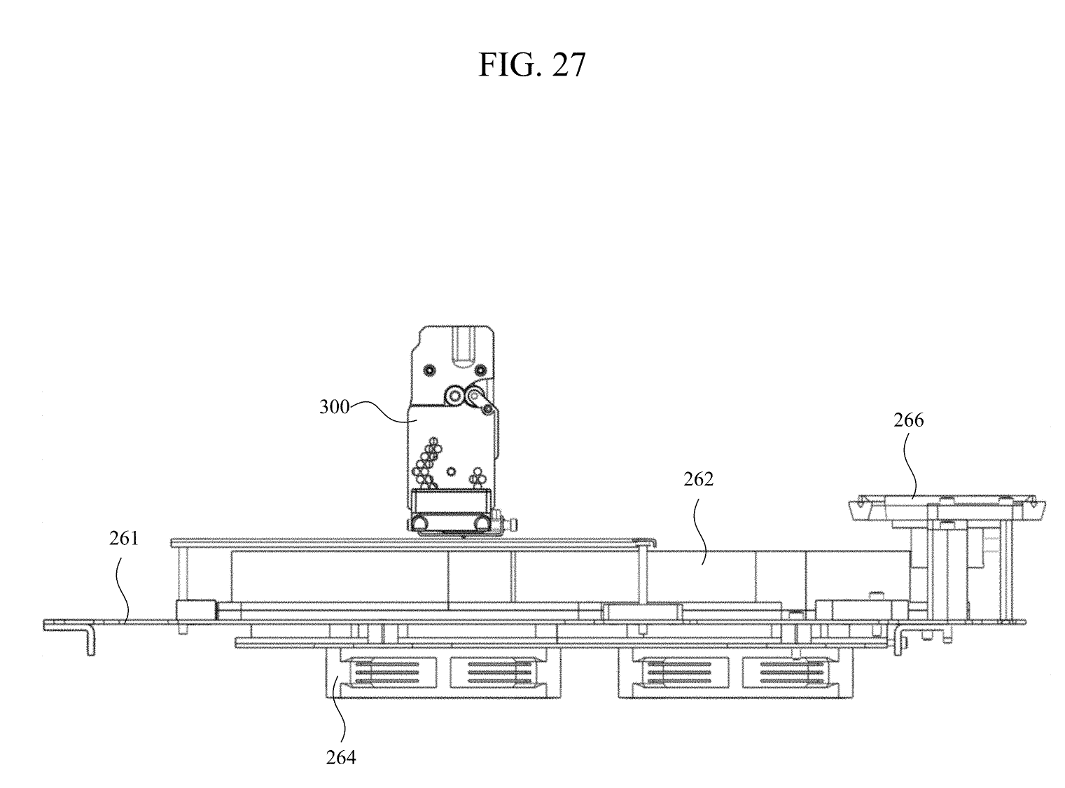

Referring to FIG. 20 to FIG. 27, in some embodiments the dispensing system comprises a containment system and a support structure configured to contain a dispensed material and determine the mass of a dispensed material from the dispenser apparatus. The containment system comprises a primary base 261 formed with a plurality of holes allowing a plurality of prongs 268 of the support structure to penetrate through. A bin 262 is disposed on a top surface of the primary base 261, and configured to accommodate a screen 263. The screen 263 is configured to filter a dispensed material using a first fan 264. In the present embodiment, the screen files particles at the micron scale; however, the present disclosure is not limited thereto.

The first fan 264 draws air through the screen 263. In the present embodiment, the containment system comprises a plurality of fans 264 that draw air in through the screen 263.

In some embodiments, a printing plate 265 is disposed above the bin 262, formed in a ‘T’-shape, and is configured to be a target dispensing location. However, the present disclosure is not limited thereto. For instance, in another embodiment the printing plate 265 is formed in an ‘I’ or plate shape.

The support structure comprises a balance 267 configured to determine the mass of a dispensed material. Disposed on the balance are the plurality of prongs 268 which penetrate the plurality of holes of the primary base 261 and support the printing plate 265. During a dispensing operation, the support prongs 268 are disengaged from the printing plate 265. After a dispensing operation, the support prongs 268 lift the printing plate 265 so that the printing plate 265 is suspended from the bin 262 and is capable of being measured by the balance 267. The lifting is provided by a series of motors (not shown) and/or belts. In some embodiments, the balance continuously determines, or determines on an automated recurring basis, the mass of dispensed material. The dispenser 100 ceases operation when the mass of dispensed material is equal to a predetermined value.

In some embodiments, a docking station 266 is disposed on the containment system and accommodates the end cap 280. The end cap 280 is magnetically disposed on the bottom surface of the lower base 2 of the dispenser 100. When a dispenser 100 is not in use, the end cap 280 covers the gate 13 and gate hub 14 so that no material can be dispensed. The end cap 280 is removed by the docking station 266 when the dispenser 100 is selected for operation. When the dispenser 100 is selected for operation, the dispenser moves over the docking station 266 and the end cap 280 is removed. After a dispensing operation ceases, the dispenser 100 moves over the docking station 266 again, and the end cap 280 is reapplied.

Accordingly, a dispensing system according to an exemplary embodiment of the present disclosure achieves the advantages of a reduction in the total number of actuators required for operation, reduction in the risk of contamination of the dispensing material, eliminates galling and micro-impacts using as few moving parts as possible, designed for simplicity and reduced cleaning and maintenance, and is modular wherein a single dispensing station may operate a plurality of dispensers.

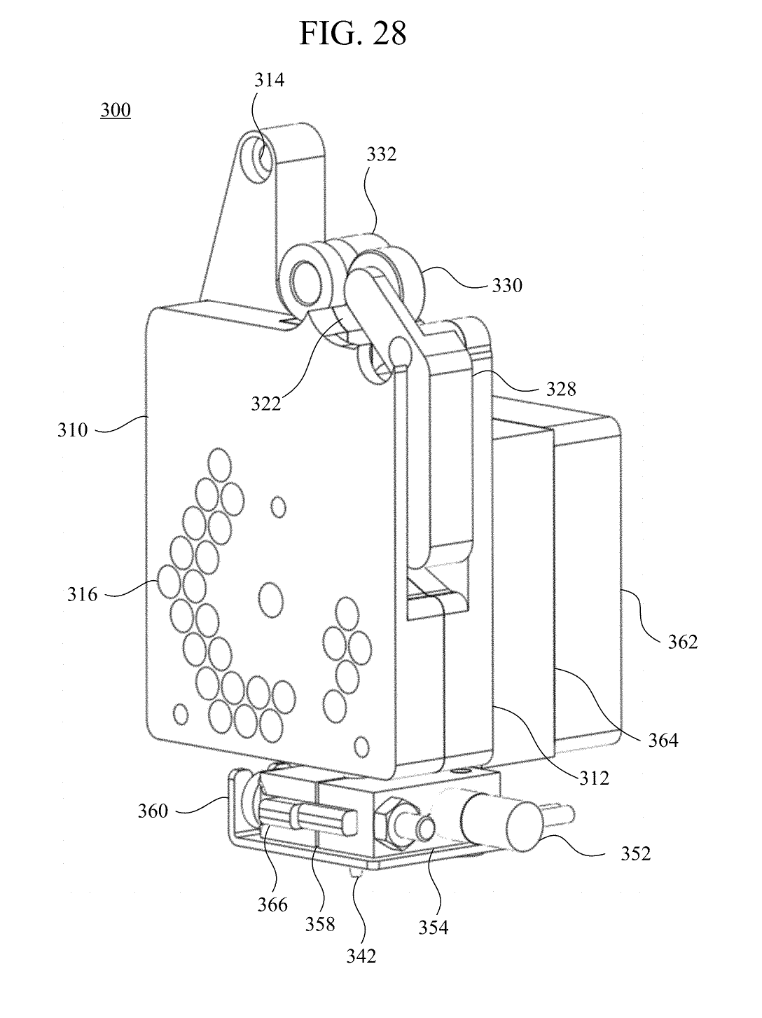

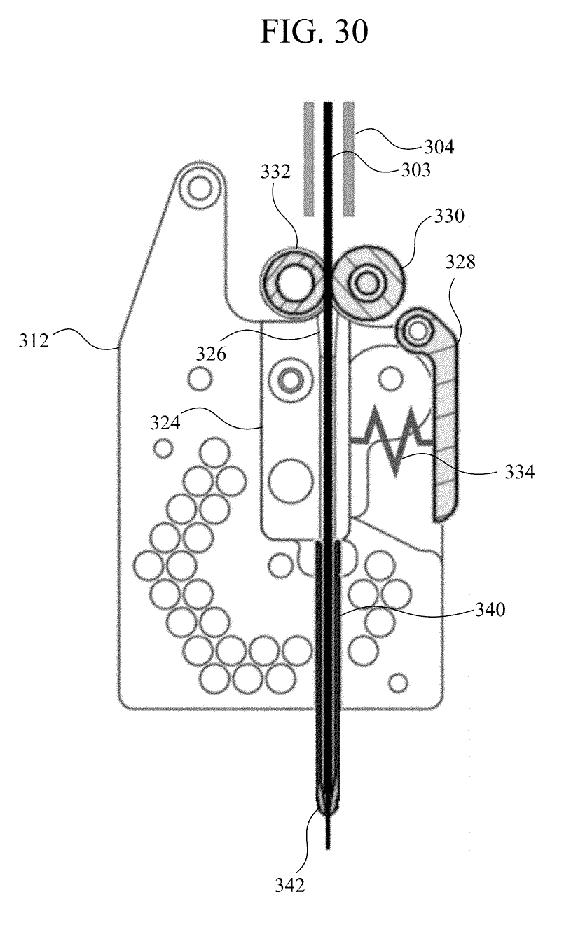

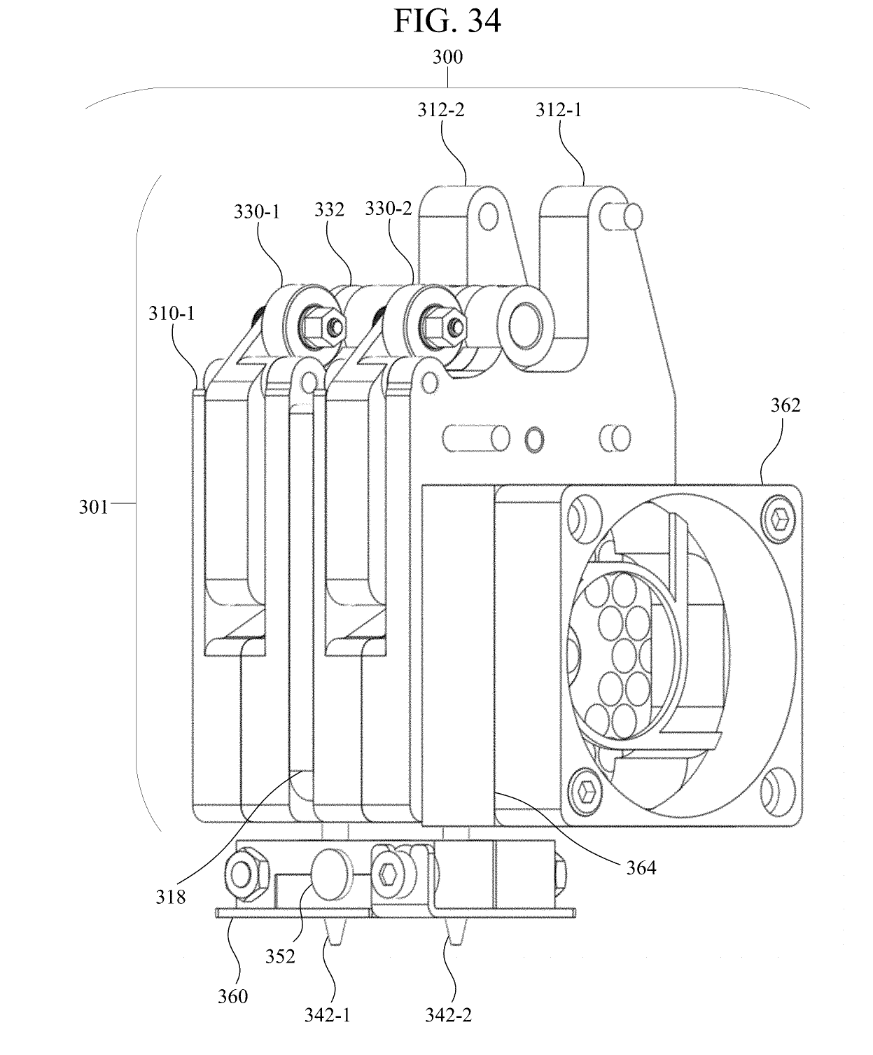

Another aspect of the present disclosure is directed to providing a filament extruder 300 (e.g., a filament extruder that is attached to a three-dimensional printer). Accordingly, the filament extruder of the present disclose is operated and actuated by standard processes of conventional three-dimensional printers. The filament extruder is configured to extrude a plurality of filament solutions (e.g., filament 303 of FIG. 30) on a target area. In some embodiments, each filament extruder 300 includes a plurality of extruder sets 301 (e.g., extruder set 301 of FIG. 34). Each extruder set 301 includes a first extruder 302-1 and a second extruder 302-2, that each includes at least an extruder portion 320 and a hot end portion 350. However, in some embodiments each extruder set 301 includes one extruder 302 (e.g., extruder 300 of FIG. 28), three extruders, four extruders or more. Various configurations and arrays of extruders 302 and/or extruder sets 301 will be described in more detail infra. In some embodiments, extrusion of the plurality of filament solutions is performed simultaneous. In some embodiments, the extrusion is performed in predetermined sequences and/or combinations of a first plurality of filaments in the plurality of filaments.

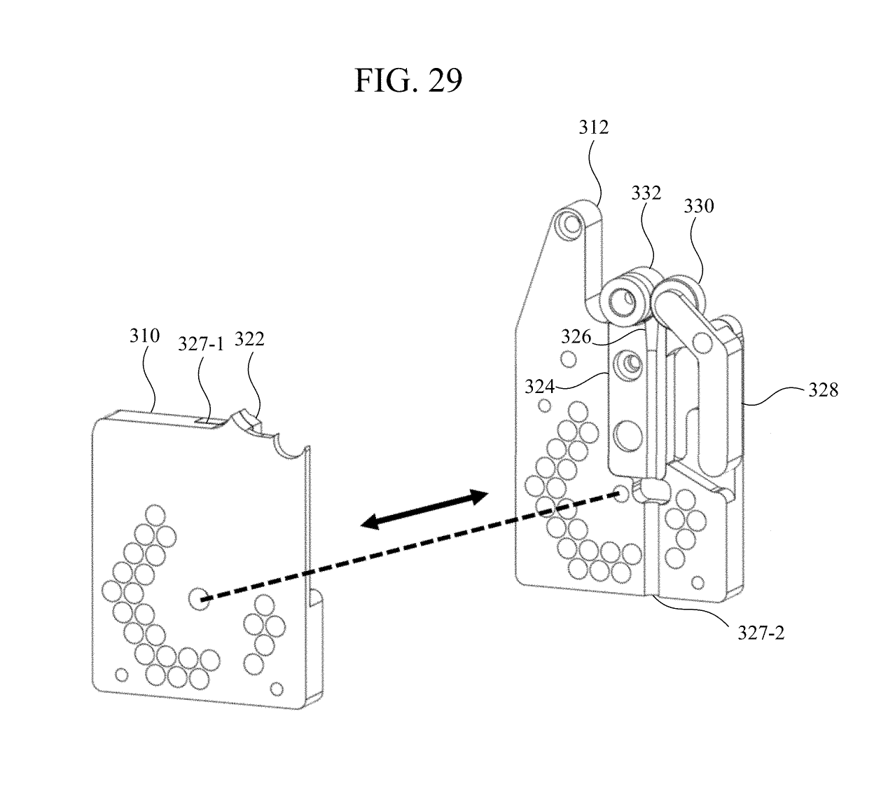

Referring to FIG. 28 through FIG. 31, the extruder portion 320 of each extruder 302 drives a filament 303 in the plurality of filaments from a supply reservoir to the hot end portion 350. In some embodiments, a body of the extruder portion 320 includes a first plate 310 and a second plate 312. The first plate 310 and the second plate 312 are removeably coupled together, enabling access to an interior of the extruder portion 320. If the first plate 310 and the second plate 312 are combined, a first housing is formed to hold various components of the extruder portion. In some embodiments, the first plate 310 and the second plate 312 are formed with a plurality of ventilation holes 316. However, in some embodiments the first plate 310 and the second plate 312 are formed omitting the ventilation holes 316. In some embodiments, one of the first plate 310 or the second plate 312 is formed with the ventilation holes. Moreover, in some embodiments the ventilation holes 316 are one or more slits, a plurality of fins, or a similar shape that allows for a sufficient flow of air through the extruder portion. The shape and a number of ventilation holes 316 can vary from one embodiment to another embodiment.

In some embodiments, a locating hole 314 is disposed on the second plate 312. The locating hole 314 fixes the filament extruder 300 to the three-dimensional printer. In some embodiments, the locating hole 314 is disposed on the first plate 310. Furthermore, in some embodiments the locating hole 314 is a locating pin or similar device to couple to filament extruder of the present disclose to the three-dimensional printer. Disposition of the locating hole is not limited to a specific portion of the extruder portion. For instance, in some embodiments the locating hole is disposed on an upper portion of either plate, an upper portion of both plates, a lower portion of either plate, a lower portion of both plates, a middle portion of either plate, and/or a middle portion of both plates.

Each of the first plate 310 and the second plate 312 is formed with a first recessed groove 327-1 that accommodates a first insert 322 and a second insert 324, respectively. In some embodiments, the first insert 322 and the second insert 324 are removeably coupled to the first plate 310 and the second plate 312. In some embodiments, one of the first insert or the second insert 322 is removeably coupled to either the first plate 310 or the second plate 320, respectively. Furthermore, in some embodiments the first insert 322 and the second insert 324 are integrally formed with the first plate 310 and the second plate 312, respectively. The first plate 310 and the second plate 312 are each formed with a second recessed groove 372-2 that accommodates a needle tube 340. Moreover, in some embodiments, the first plate 310, the second plate 312, or a combination thereof have a heat sink 362 and/or a fan 364 disposed on a surface thereof. The heat sink 362 and the fan 364 are disposed is a variety of push and/or pull combinations in order to control a flow of air, and thus temperature, of the extruder portion 320.

The first insert 310 and the second insert 321 form a channel 326 when combined. The channel 326 receives a filament 303. To assist in receiving the filament 303, a first opening of the channel 326 is formed as a funnel. However, the present disclosure is not limited thereto. For instance, in some embodiments the channel 326 is formed as a straight channel, is formed with a plurality of protruding ribs that prevent the filament 303 from withdrawing up the channel, or is formed as a conical channel.

An assembly, that includes a lever 328, a bearing 330, a pinch wheel 332, and a spring 334, drives the filament 303 into the extruder portion 320. The pinch wheel 332 is disposed proximate to a first side of the first opening of the channel 326. Accordingly, the bearing 330 is disposed proximate to a second side of the first opening of the channel 326. The lever 328 is disposed on a first side the extruder portion 320 and coupled to the bearing 330. The lever 328 has at least a first position in which the lever 328 drives the bearing 330 against the filament 303 that is between the bearing 330 and the pinch wheel 332, which causes the filament 303 to traverse the channel 326 towards the needle tube 340. Similarly, the lever 328 has at least a second position in which the bearing 330 is disengaged from the pinch wheel 332 and the filament 303, preventing the filament 303 from traversing the channel 326 towards the needle tube 340. In some embodiments, the lever 328 includes a plurality of positions. Each position in the plurality of positions is associated with an extrusion rate of the filament 303. For instance, in some embodiments each position in the plurality of positions is associated an extrusion rate of from 0 millimeters per second (mm/s) to 50 mm/s. In some embodiments, each position in the plurality of positions is associated with an extrusion rate of from 0 mm/s to 25 mm/s. In some embodiments, each position in the plurality of positions is associated with an extrusion rate of from 0 mm/s to 50 mm/s in intervals of 0.1 mm/s. In some embodiments, the first position is an ON state of the extruder and the second position is an OFF state of the extruder. For instance, in some embodiments, e.g., when the first position is the ON state, an external motor is activated which rotates the pinch wheel 332, the bearing 320, or a combination thereof.

In some embodiments, the pinch wheel 332 includes a groove for each filament the pinch wheel is configured to guide. In some embodiments, the pinch wheel 332 of each extruder portion 320 is integrated as a single common pinch wheel 332. For instance, referring to FIG. 34, the pinch wheel 332 is a double-pinch wheel 332, which allows a single motor to actuate the extruder set 301.

A spring 334 is disposed between the lever 328 and the housing that is the first plate 310 and the second plate 312. In some embodiments, the spring 334 is disposed on the first insert 322, the second insert 324, or both the first insert and the second insert. The spring 334 actuates the lever 328 between the plurality of positions (e.g., the first position and the second position). In some embodiments, the spring 334 is an electro-permanent magnet. In some embodiments, the spring 334 is integrally formed with the lever 328 (e.g., as an elastic portion).

In some embodiments, a guide tube 304 is disposed above the extruder portion 320. The guide tube 304 guides the filament 303 between the pinch wheel 332 and the bearing 330. In some embodiments, the guide tube 334 is formed from a plastic (e.g., polytetrafluoroethylene (PTFE)), a metal or metal alloy (e.g., aluminum, stainless steel, titanium).

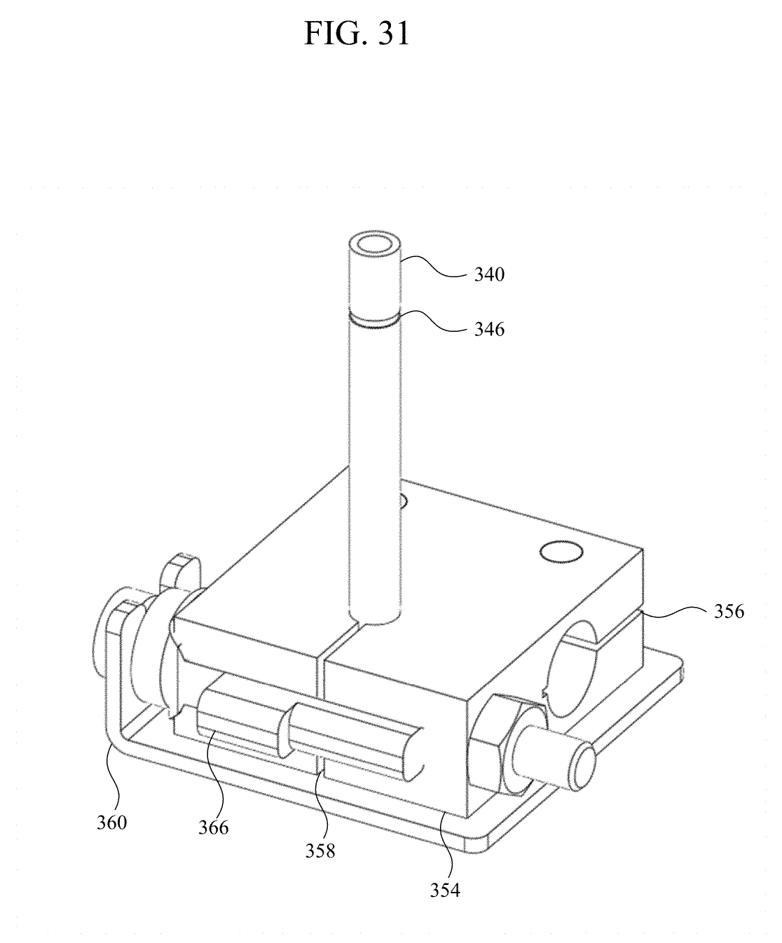

Referring to FIGS. 30 and 31, the extruder portion 320 is coupled to the hot end portion 350 via the needle tube 340. The needle tube 340 is, in part, responsible for transferring heat to the filament 303 as well as guiding a course and a flow of various phases of the filament. In some embodiments, a nozzle 342 is disposed on a bottom surface of the needle tube 340 or is integral to the needle tube. The nozzle 342 is configured to lay the filament 303 on a portion of a target surface when the hot end portion 350 melts the filament. In some embodiments, the nozzle 342 has a shape that is conical or comes to a point (e.g., a sharp point or a curved point. To allow for cleaning, the needle tube 340 is removeably coupled to the first insert 322, the second insert 324, the first plate 310 and the second plate 320. In some embodiments, the needle tube 340 includes a stopper which couples to the needle tube to the extruder portion 320. The stopper is a device which protrudes from the needle tube and holds the needle tube 340 to the first plate 310 and/or the second plate 312 in order to prevent the needle tube from falling out of the extruder portion 320. In some embodiments, the needle tube 340 includes a groove 346 that accommodates the stopper. For instance, in some embodiments the groove 346 accommodates a ring type (e.g., O-ring) stopper or a flexible pincer type stopper. In some embodiments, a spacer 318 is removeably disposed interposing between the first extruder 302-1 and the second extruder 302-2. The spacer 318 is configured to reduce a distance between the first plate 310 and the second plate 312 of each of the first extruder 302-1 and the second extruder 302-2. In some embodiments, the spacer 318 reduces a distance between the first plate 310 and the second plate 312 such that the stopper of the needle is engage with the first plate 310 and the second plate 312.

In some embodiments, the needle portion 340 is removeably coupled to the hot end portion 350 through a clamp. In some embodiments, the clamp includes a through hole in which the needle tube 340 is inserted and a second gap 358. When a force is applied to the second gap 358, a size of the through hole is reduce and movement of the needle tube 340 is restricted, preventing the needle tube 340 from being withdrawn from the hot end portion 350. In some embodiments, the clamp is a bolt. In some embodiments, there is a one-to-one relationship with each clamp and each needle tube, or similarly a one-to-many relationship.

The hot end portion 302 includes a heater 352, a heater block 354, and a thermistor 366. Encompassing at least a portion of each needle tube 340 is the heater block 354, which melts each filament 303. In some embodiments, the heater block 354 is a single piece. Moreover, in some embodiments the heater block 354 includes a first portion 354-1 and a second portion 354-2. The first portion 354-1 in combination with the second portion 354-2 holds the heater 352 to the hot end portion 350. The second portion 354-2 is removeably coupled to the first portion 354-2 to allow removal of the heater 352 from the hot end portion 350. In some embodiments, the second portion 354-2 is magnetically coupled to the first end portion 354-1, screwed to the first end portion, or slideably coupled to the first end portion. Preferably, the coupling is flush such that there is no interference with a distance between the target surface and each nozzle 346.

The heater 352 is configured to heat the heater block 354. The heater 352 is disposed in such a way that heat is uniformly transferred to each needle tube 340 and/or nozzle 346. In some embodiments, the heater 352 and the heater block 354 are integrally formed. In some embodiments, the heater 352 is a cartridge heater or a tubular heater. In some embodiments, there is one or more heaters 352 for each hot end portion 350.

In some embodiments, the thermistor 366 is disposed on a surface of the heater block 354. The thermistor 366 detects a temperature of the filament 303 and/or the heater block 354.

A shroud 360 houses the hot end portion 350 and shields extruded filament 303 from contamination and/or external interference. In some embodiments, the shroud 360 is coupled to the heater block 354. In some embodiments, the coupling is through the clamps which hold the needle tube 340 to the hot end portion 350. The shroud 360 is a continuous piece such that it is removed from the filament extruder 300 and easily cleaned.

In some embodiments, the bearing 330, the pinch wheel 332, the spring 334, the first insert 322, the second insert 324, and/or the shroud 360 are formed of 316 stainless steel or a similar material suitable for food grade and/or pharmaceutical grade manufacturing as determined by various jurisdictions and guidelines.

In some embodiments, the needle tube 340 is formed of 304 stainless steel. In some embodiments, the needle tube 340 is formed of a material other than 304 stainless steel such as titanium, aluminum, stainless steel 316.

In some embodiments, the first plate 310, the second plate 312, the lever 328, and/or the heater block 354 are formed of aluminum. In some embodiments, the first plate 310, the second plate 312, the lever 328, and/or the heater block 354 are formed of an aluminum alloy, or a material or alloy other than aluminum or aluminum alloy such as titanium, stainless steel 316, stainless steel 304, and/or plastic.

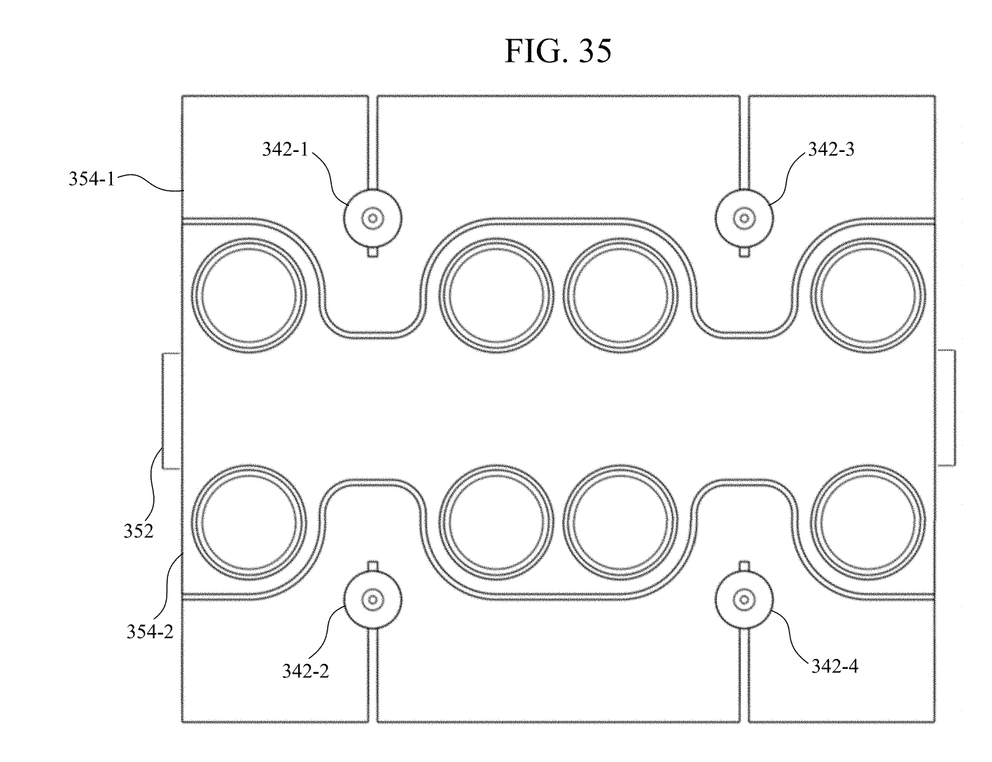

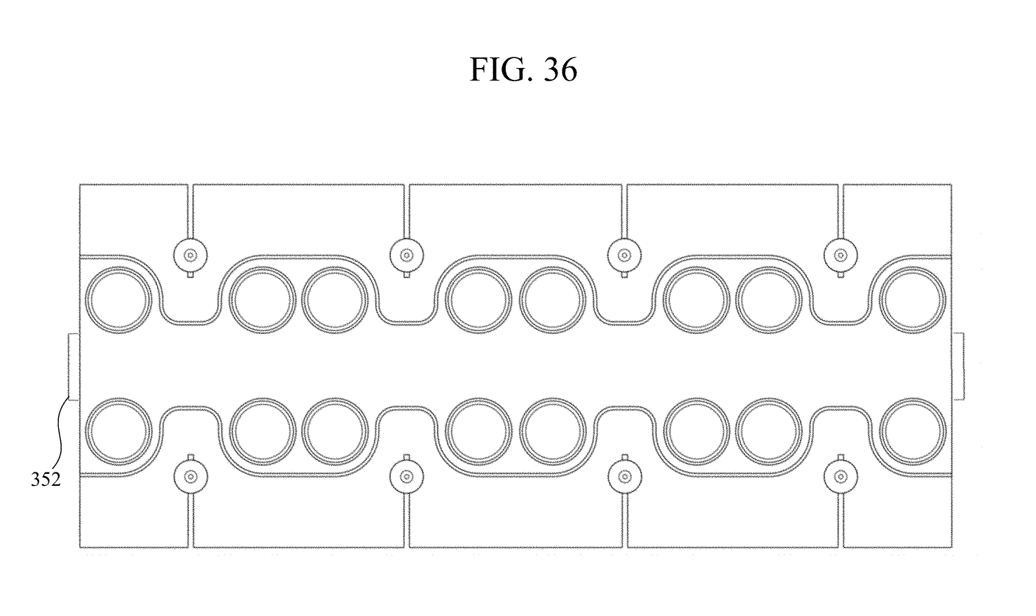

Referring to FIG. 35, an embodiment of a filament extruder 300 is depicted which extrudes four filaments 303 from four corresponding nozzles 342-1, 342-2, 342-3, and 342-4 utilizing a single hot end portion 350 including one heater 352. Referring to FIG. 36, an embodiment of a filament extruder 300 is depicted which extrudes eight filaments 303 from eight corresponding nozzles 346 utilizing a single hot end portion 350 including one heater 352. In some embodiments, the plurality of extruder sets 301 is disposed in a circular array. In some embodiments, the plurality of extruder sets 301 is disposed in a rectangular array.

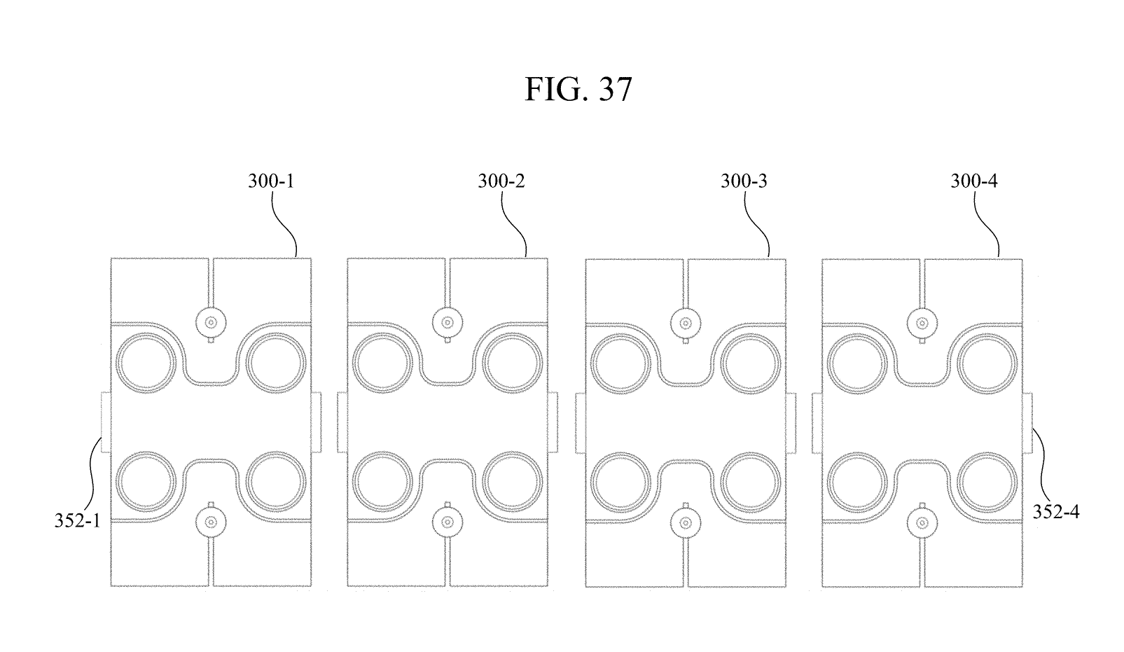

Referring to FIG. 37, in some embodiments a plurality of filament extruders 300 are orchestrated in unison, which can produce the same effect as the embodiments previously describe by FIGS. 35 and 36. A plurality of arrays and layouts are available for the plurality of filament extruders, such as a rectangular array or a circular array.

Accordingly, a filament extruder according to an exemplary embodiment of the present disclosure achieves the advantages of an apparatus that is easily assembled and disassembled for cleaning and maintenance, has a limited heated volume, and is capable of dispensing pharmaceutical grade material. A filament extruder according to an exemplary embodiment produces a plurality of products simultaneously without having to reconfigure a three-dimensional printer.

Further aspects of the present disclosure are directed to providing a method of making a capsule 400 that includes a plurality of compartments. Each compartment in the plurality of compartments 420 encapsulates a material 460. In some embodiments, an extrusion of a filament solution is initiated (e.g., an extrusion process including the filament extruder of the present disclosure). Accordingly, the filament solution is then laid down to form a base of the capsule 400. The bases of the capsule can include a plurality of shapes such as a dome, a planar surface, or a ring. A first compartment 420-1 in the plurality of compartments 420 is formed. This forming creates a first barrier wall 440-1 having a first predetermined release time. A second compartment 420-2 in the plurality of compartments 420 is formed. This second forming creates a second barrier wall 440-2 having a second predetermined release time. The first compartment 420-1 is filled with a first material 460-1. The second compartment 420-2 is filed with a second material 460-2. The plurality of compartments 420 are then sealed thereby forming the capsule 400 with a first sealed compartment 420-1 and a second sealed compartment 420-2.

In some embodiments, a first compartment 420-1 in the plurality of compartments 420 is formed and then filled with a first material 460-1 from a selection of materials 460. The first compartment 420-1 is then sealed, preventing contamination of the first material 460-1. A second compartment 420-2 in the plurality of compartments 420 is then formed and then filled with a second material 460-2. The second compartment 420-2 is then sealed, preventing contamination of the second material 460-2. This process may reiterate for any number of desired compartments in each capsule.

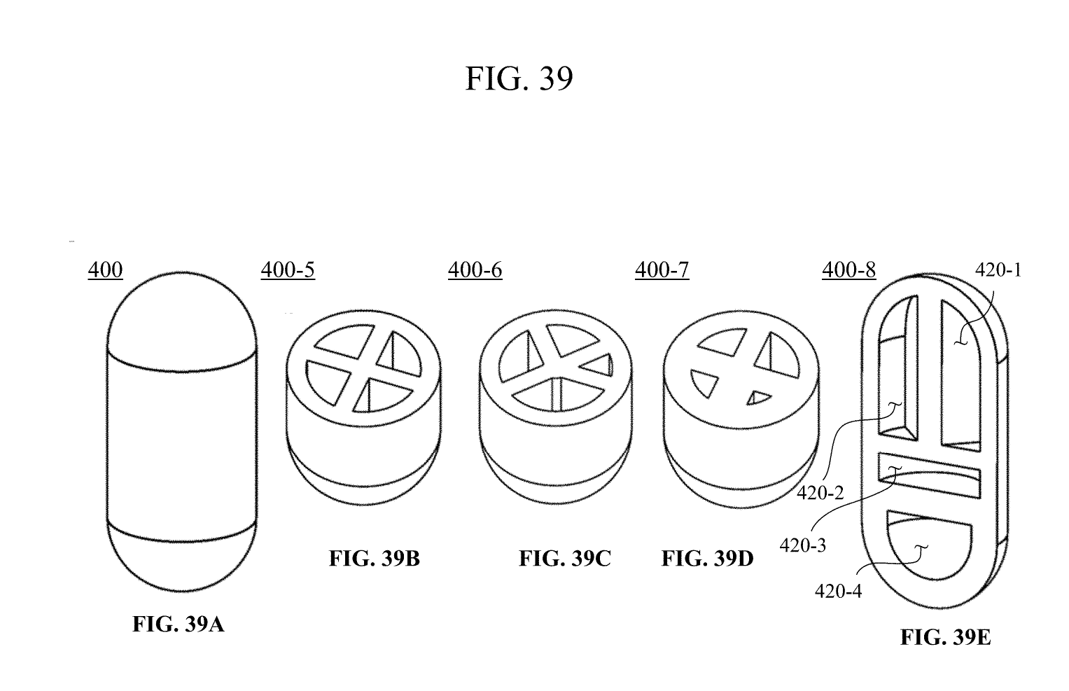

In some embodiments, the method of making a capsule 400 comprises forming the first compartment 420-1 and the second compartment 420-2 concurrently. For instance, in some embodiments the capsule 400 includes a plurality of compartments 420 that are separated by a plurality of barrier walls 440 that extend vertically. Accordingly, each compartment 420 in the plurality of compartments 420 is formed simultaneous to every other compartment 420 in the plurality of compartments 420 of the capsule 400 (e.g., capsule 400-5 through 400-7 of FIG. 39). In some embodiments, the first compartment 420-1 and the second compartment 420-2 are sequentially formed. For instance, in some embodiments the capsule 400 includes a plurality of compartments 420 that are separated by a plurality of barrier walls 440. Accordingly, each compartment 420 in the plurality of compartments is formed sequentially. In some embodiments, the concurrence or sequencing of the forming of the plurality of compartments 420 is determined by an orientation and design of the capsule 400 being formed.

In some embodiments, the method of making a capsule 400 includes performing the above steps without human intervention by an extrusion device (e.g., filament extruder 300 of FIG. 28) that is programmed with a first predetermined release time and the second predetermined release time. Moreover, in some embodiments a dispenser (e.g., dispenser 100) fills each capsule 400 with a plurality of materials 460. Accordingly, the whole method of making a capsule is performed without human intervention from the forming, filling, and sealing for each compartment in the capsule.

In some embodiments, a first portion of the base is overlaid with a first interlocking surface 490-1. The sealing forms a second interlocking surface 490-2 that joins the first interlocking surface 490-1. In other embodiments, a second portion of the base is overlaid with a third interlocking surface 490-3, and the sealing further forms a fourth interlocking surface 490-4 that joins the third interlocking surface 490-3. In still another embodiment, a third portion of the base is overload with a fifth interlocking surface 490-5, and the sealing forms a sixth interlocking surface 490-6 that joins the fifth interlocking surface 490-5. In still a further embodiment, the base comprises a first plurality of discrete interlocking surfaces 490-n1, and the sealing forms a second plurality of discrete interlocking surfaces 490-n2 and the sealing, for each respective discrete interlocking surface in the first plurality of discrete interlocking surfaces 490-n1, adjoins the respective discrete interlocking surface with a corresponding discrete interlocking surface in the second plurality of discrete interlocking surfaces 490-n2.

In some embodiments, the method of making the capsule 400 forms the capsule such that the base of the capsule comprises a first plurality of discrete interlocking surfaces 490. In such embodiments, the sealing forms a second plurality of interlocking surfaces 490 and, moreover, the sealing, for each respective discrete interlocking surface 490-1 in the first plurality of discrete interlocking surfaces 490, adjoins the respective discrete interlocking surface 490-1 with a corresponding discrete interlocking surface 490-2 in the second plurality of interlocking surfaces 490. The forming of the discrete interlocking surfaces 490 may comprise a separate injection molding process.

In some embodiments, the first predetermined release time is different than the second predetermined release time. For instance, in some embodiments, the first predetermined release time is less than five minutes and the second predetermined release time is greater than ten minutes. In another embodiment, the first predetermined release time is less than an hour and the second predetermined release time is greater than 2 hours. In yet another embodiment, the first release time is less than 30 minutes and the second release time is greater than an hour. In a further embodiment, the first predetermined release time ranges from 30 minutes to 4 hours and the second predetermined release time ranges from 6 hours to 18 hours. In some embodiments, the first predetermined release time is the same as the second predetermined release time.

In some embodiments, the first compartment 420-1 has a first barrier wall 440-1 formed of one or more vertical, horizontal, or radial walls; or the second compartment 420-2 has a second barrier wall 440-2 formed of one or more vertical, horizontal, or radial walls. According to the present disclosure, in another embodiment, the barrier walls 440 and the compartments 420 are formed in any number, size, and combination of vertical, horizontal, or radial walls.

Depending on the end design structure and goal, the plurality of barrier walls 440 are formed radially during a single extrusion process.

In some embodiments, the first material 460-1 is different than the second material 460-2 (e.g., the first material includes a first pharmaceutical composition and the second material includes a second pharmaceutical composition). In another embodiment, the first material 460-1 is the same as the second material 480-2. For instance, in some embodiments the first material 460-1 and the second material 460-2 are a same material, but release rates of the containers 420 therein are different.

According to the present disclosure, the first predetermined release time is determined by a first characteristic of the first barrier wall 440-1. The first characteristic of the first barrier wall 440-1 comprises a thickness of the wall or porosity of the wall. Similarly, the second predetermined release time is determined by a first characteristic of the second barrier wall 440-2. The first characteristic of the second barrier wall 440-2 comprises a thickness of the wall or porosity of the wall. In some embodiments, the first barrier wall has a thickness less than 0.1 mm and the second barrier wall has a thickness greater than 0.2 mm. In another embodiment, the first barrier wall has a thickness less than 0.5 mm and the second barrier wall has a thickness greater than In yet another embodiment, the first barrier wall has a thickness that ranges from 0.01 mm to 0.5 mm and the second barrier wall has a thickness that ranges from 0.6 mm to 1 mm.

In some embodiments, the first characteristic of the first barrier wall 440-1 comprises a pH sensitive material. Similarly, the second predetermined release time is determined by a first characteristic of the second barrier wall 440-2. The first characteristic of the second barrier wall 440-2 comprises a different pH sensitive material, so that the second barrier wall 440-2 decomposes at a pH different than the first barrier wall 440-1.

In some embodiments, the first sealed compartment 420-1 has a volume that is different than the second sealed compartment 420-2. In another embodiment, the first sealed compartment 420-1 has a volume that is the same as the second sealed compartment 420-2. In another embodiment, the first sealed compartment has a volume less than 0.5 mL and the second sealed compartment has a volume greater than 0.6 mL. In yet another embodiment, the first sealed compartment has a volume less than 0.05 mL and the second sealed compartment has a volume greater than 1 mL. In a further embodiment, the first sealed compartment has a volume that ranges from 0.01 mL to 0.2 mL and the second sealed compartment has a volume that ranges from 0.2 mL to 0.5 mL.

Other embodiments of the present disclosure comprise the method of making a capsule 400, wherein the plurality of compartments 420 comprises three or more compartments 420-3 to 420-n.

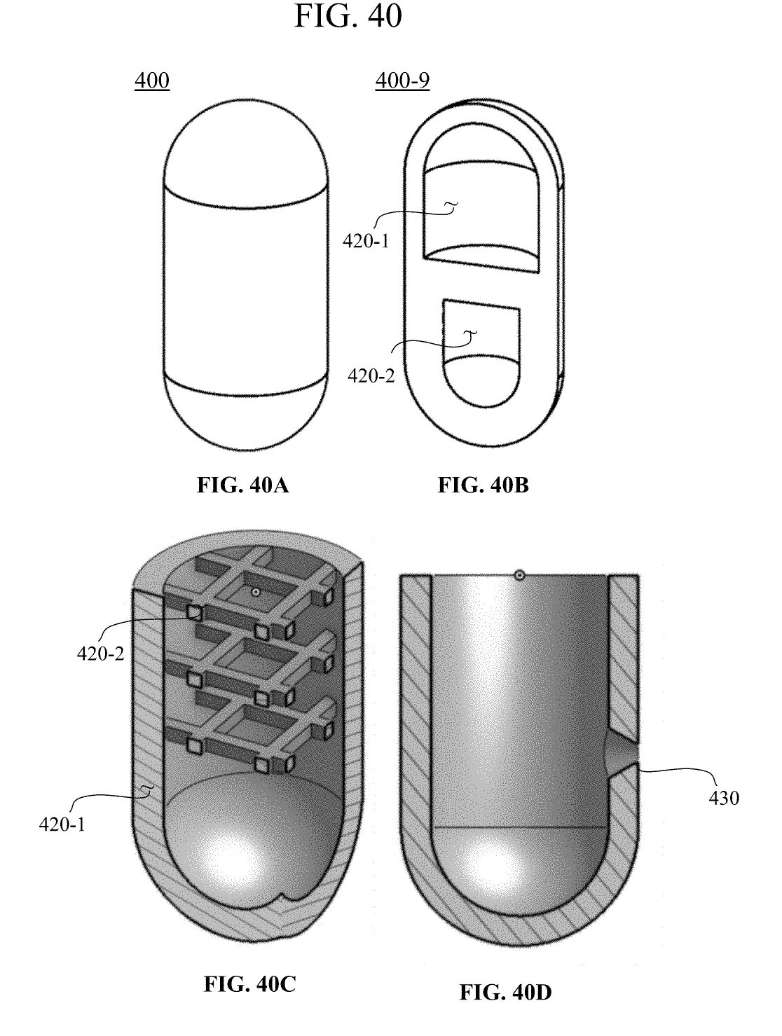

Using extrusion processes (e.g., extrusion of a filament solution using a three-dimensional printer) to form the capsules 400 of the present disclosures allows for the design of complex structures and architectures that are otherwise too costly or impossible to achieve with other processes, such as injection molding. For instance, in some embodiments the plurality of compartments 420 is a plurality of concentric compartments (e.g., concentric spheres, concentric domes, concentric cylinders, or concentric rectangles). Referring to FIG. 40C, in some embodiments a capsule 400 includes a first compartment 420-1 and a second compartment 420-1 that is formed as a bridge structure spanning from a first internal portion of the capsule to at least a second internal portion of the capsule. In some embodiments, the external walls of the capsule 400 are formed with one or more undercuts. In some embodiments, the external walls of the capsule 400 are formed with a taped through hole 430. The taped through hole is either increases in diameter when observed from an external view of the capsule or decreases in diameter when observed from the external view of the capsule. Furthermore, in some embodiments the external walls of the capsule are formed as 90-degree angles.

The shape of the capsule 400 is defined by the shape and number of compartments 420. However, the capsule 400 may comprise a predetermined shape that is stored in a controller or a software (not shown). The present predetermined shape comprises an hourglass, an ellipsoid, or a cylinder with hemispheres. However, the present disclosure is not limited thereto. For instance, the predetermined shape may be a non-uniform shape. In a further embodiment, the predetermined shape is asymmetric about one or more axes enabling the user to determine the orientation of the capsule 400 by touch. Also, according to the present disclosure, in another embodiment the capsule's 400 comprise a shape or a volume controlled by a predetermined algorithm or a software.

In some embodiments, the thickness of the walls ranges from 0.1 mm to 2 mm. In other embodiments, the volume of the compartments 420 ranges from 0.1 mL to 2 mL.

In another embodiment of the present disclosure, the external walls of the capsule 400 are formed with a textured surface. In some embodiments, the textured surface includes a dimple or a plurality of dimples configured to enable a user to differentiate the orientation of the capsule 400 or differentiate a first capsule capsules from a plurality of capsules. In even further embodiments, the textured surface is configured to allow the capsule 400 to bind to a food product.

Additionally, in further embodiments of the present disclosure, the capsule 400 comprises the first material 480-1 is any one from the group comprising: ethyl cellulose, hydroxypropyl cellulose, hydroxypropyl methyl cellulose, hydroxypropyl methyl cellulose acetate succinate, various types of methacrylic acid copolymers, polyethylene oxide, polyvinyl alcohol, polyvinyl alcohol-polyethylene glycol graft copolymer, and polyvinyl caprolactam-polyvinyl acetate-polyethylene glycol graft co-polymer; or the capsule 400 comprises the second material 480-2 is any one from the group comprising: ethyl cellulose, hydroxypropyl cellulose, hydroxypropyl methyl cellulose, hydroxypropyl methyl cellulose acetate succinate, various types of methacrylic acid copolymers, polyethylene oxide, polyvinyl alcohol, polyvinyl alcohol-polyethylene glycol graft copolymer, and polyvinyl caprolactam-polyvinyl acetate-polyethylene glycol graft co-polymer.

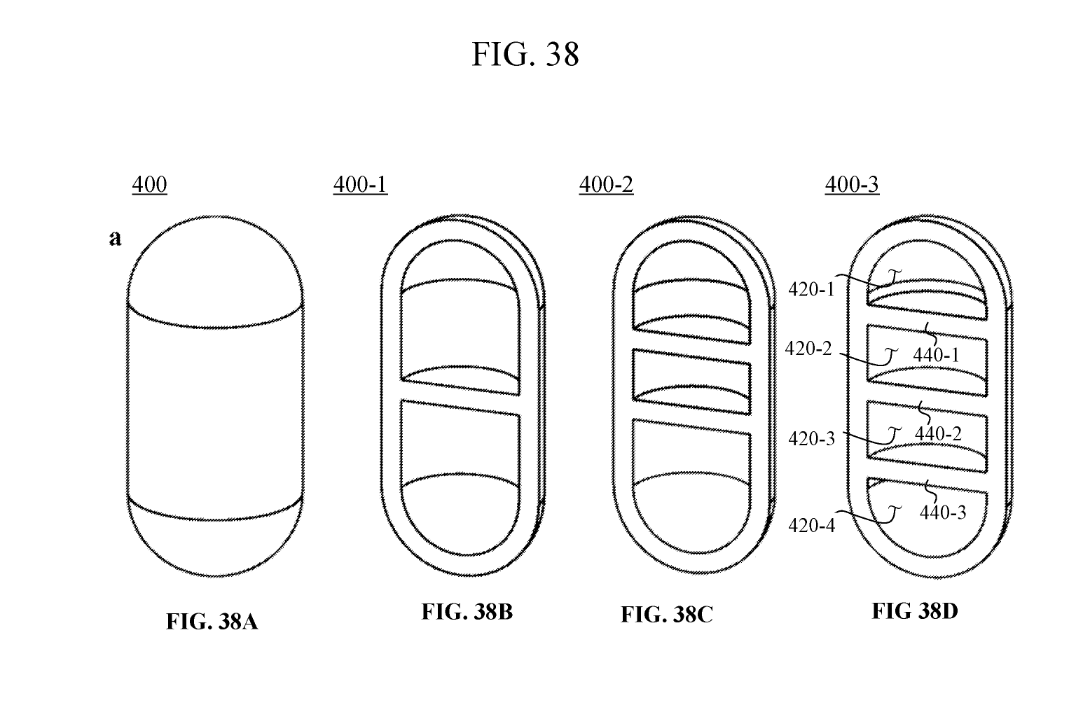

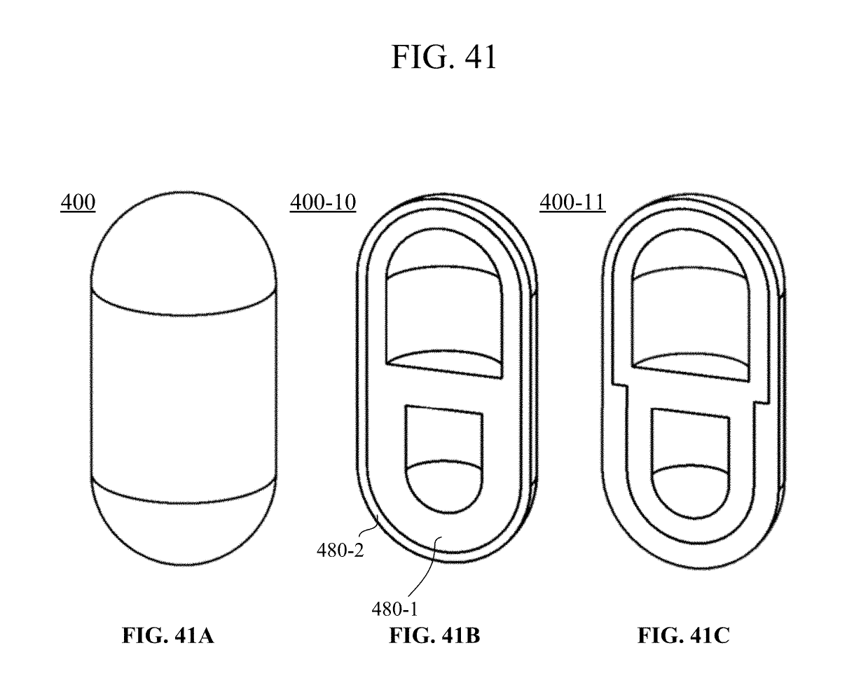

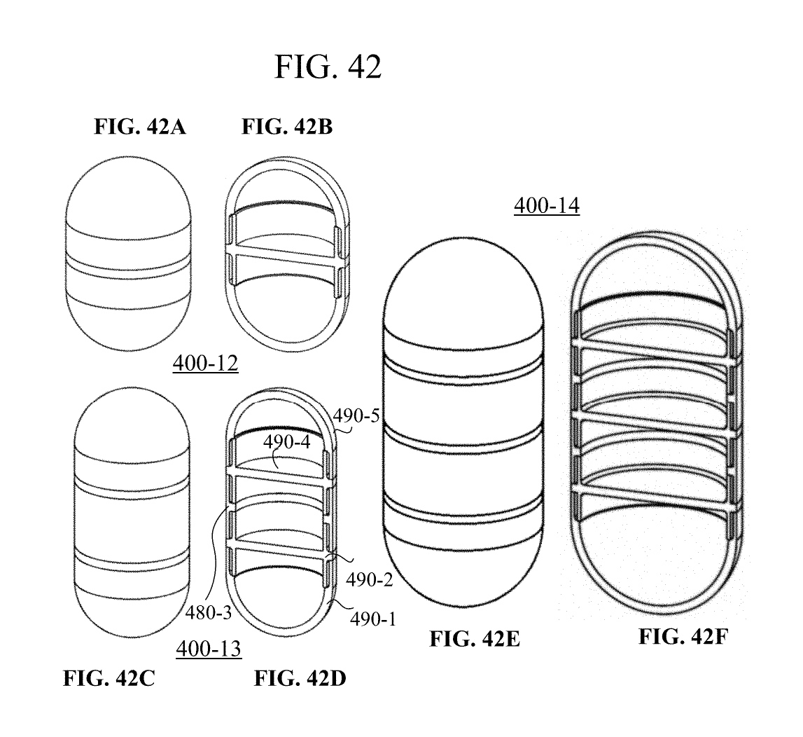

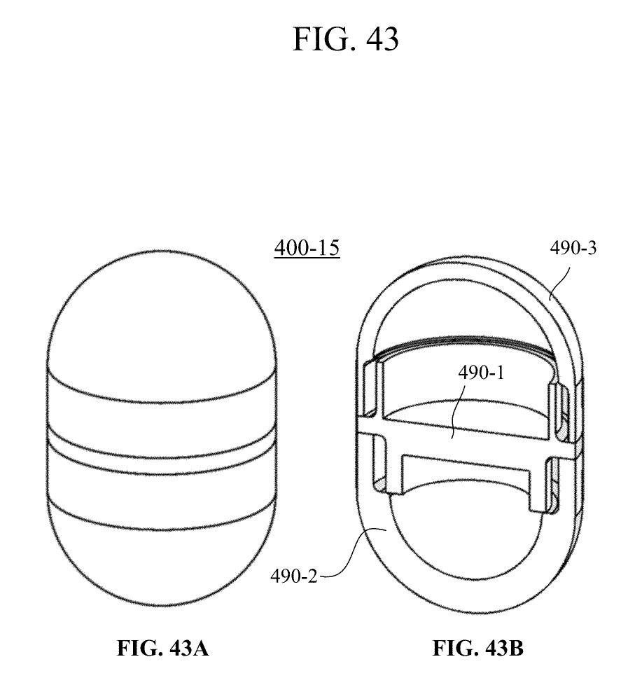

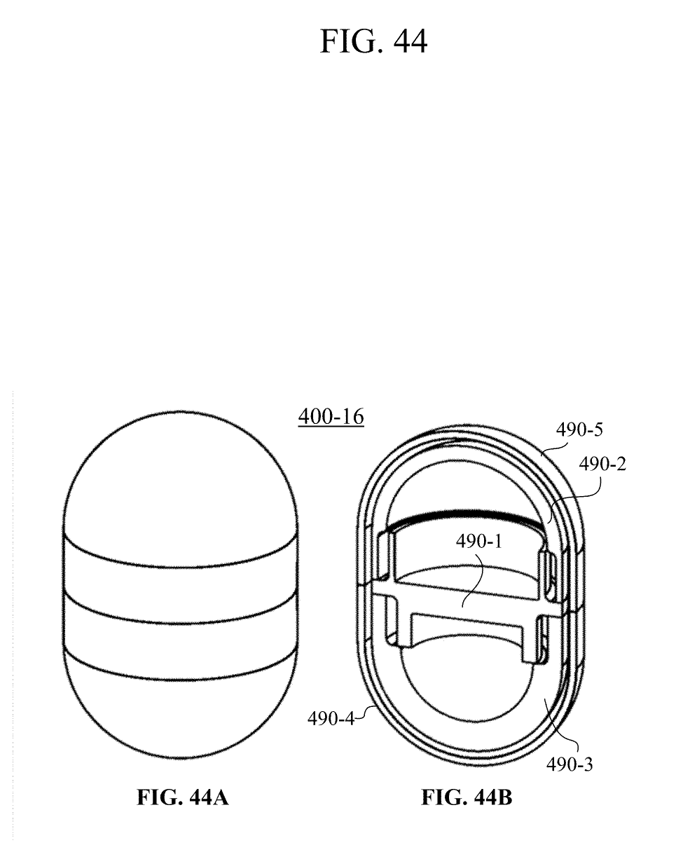

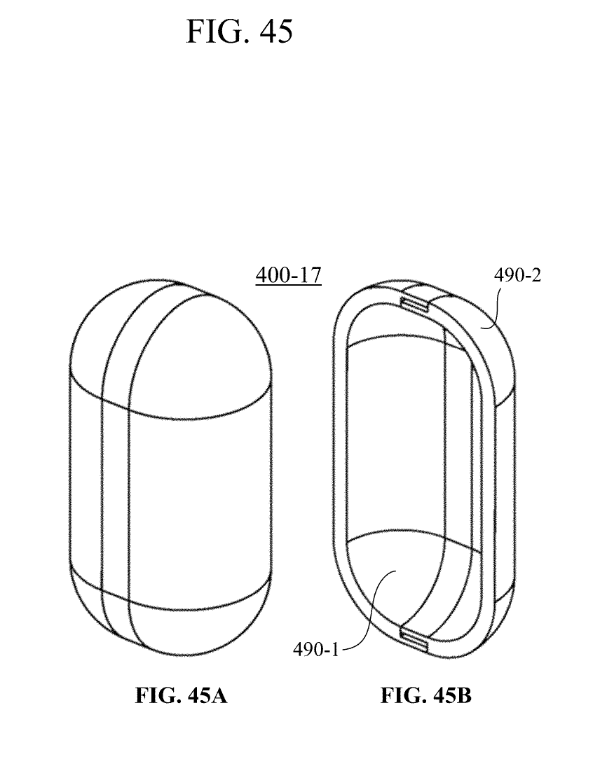

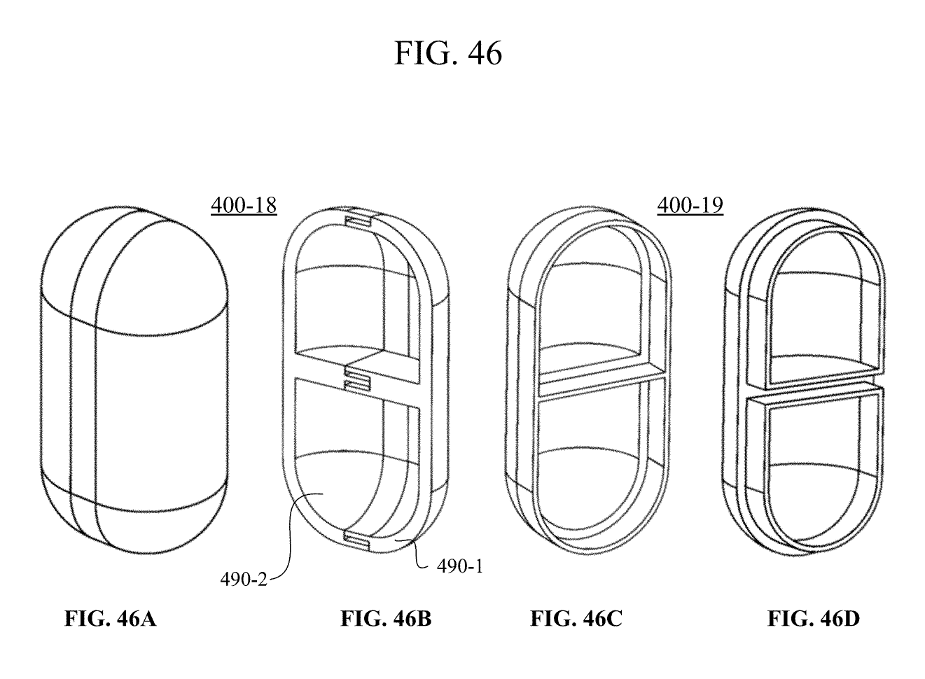

Referring to FIG. 38 to FIG. 46, exemplary embodiments of the present disclosure are provided. As shown in FIG. 38 to FIG. 41, an exemplary embodiment of the present disclosure comprises a plurality of compartments 420, a plurality of barrier walls 440, a plurality of filling material 460, or a plurality of capsule material 480. FIG. 42 to FIG. 46 depicts other embodiments of the present disclosure wherein a plurality of interlocking surfaces 490 are formed.

Accordingly, a method of making a capsule comprising a plurality of compartments, according to an exemplary embodiment of the present disclosure, addresses the deficiencies of the prior art while having the advantages of comprising a plurality of materials capable of imparting a release performance according to the thickness, composition, and design of the capsule.

Another aspect of the present disclosure is directed to providing a user interface which alieves the problems of the prior art which providing a means for the user to create a custom, single dosage capsule tailored to the user.

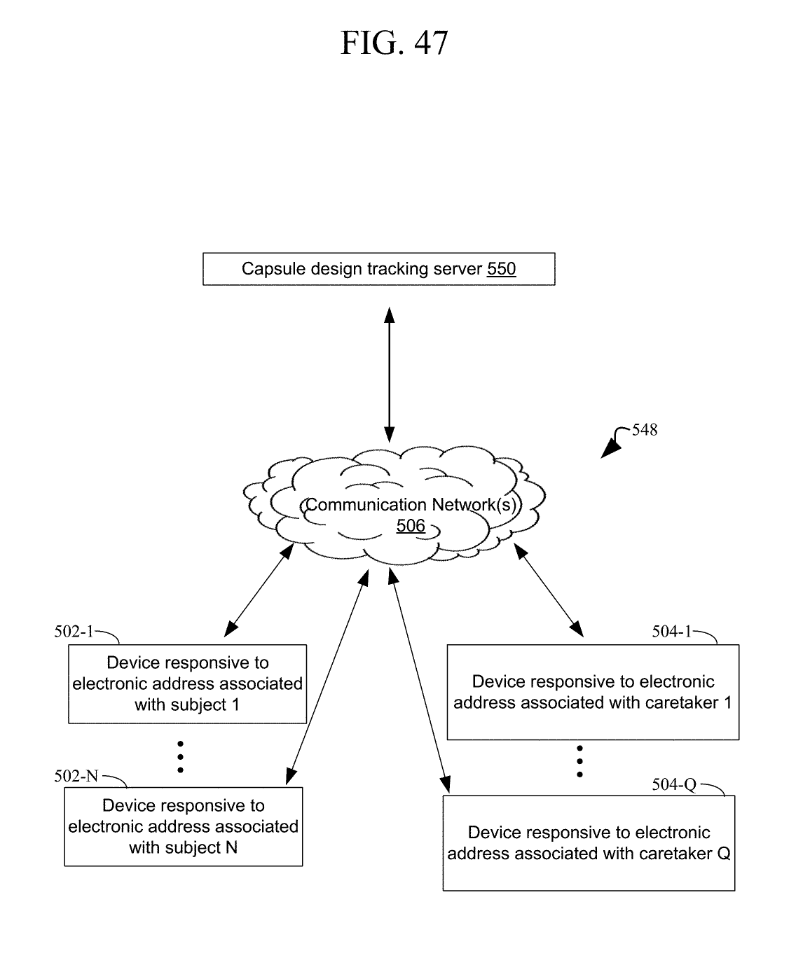

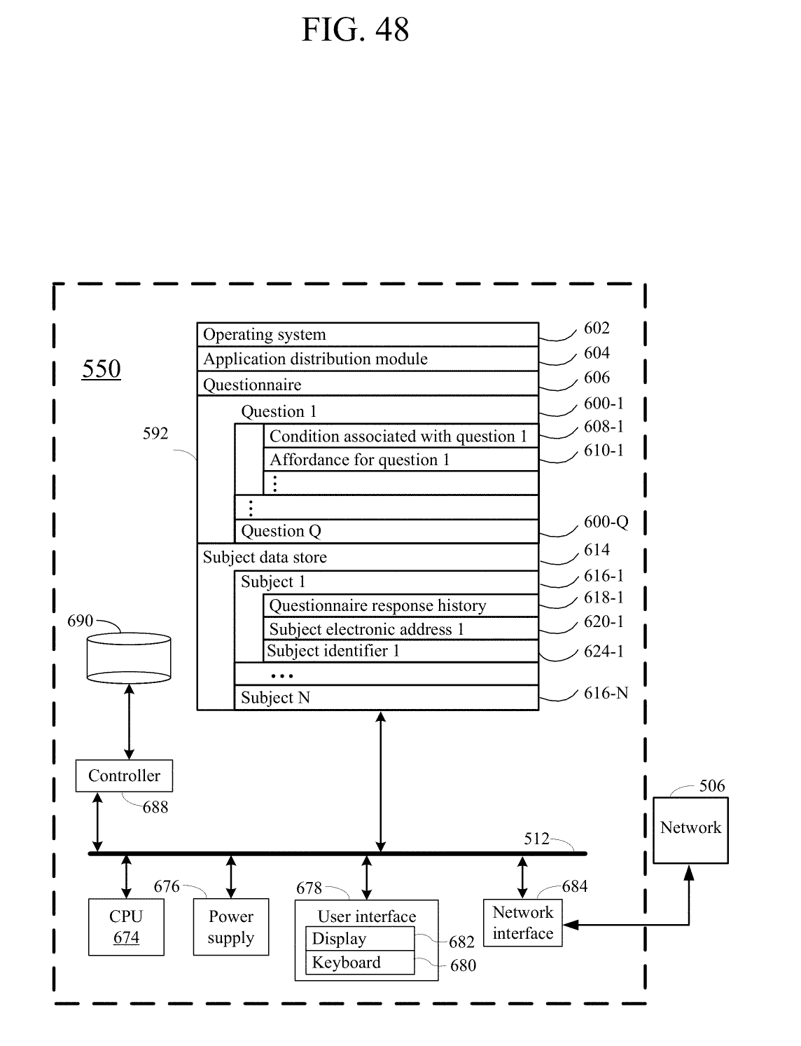

A detailed description of a system 548 for designing a tailored multi-compartment capsule in accordance with the present disclosure is described in conjunction with FIG. 47 and FIG. 48. As such, FIG. 47 and FIG. 48 collectively illustrate the topology of the system 548 in accordance with the present disclosure. In the topology, there is a capsule design tracking server 550 (FIG. 47 and FIG. 48), devices 502 responsive to electronic addresses associated with subjects to be monitored (FIG. 47), and devices 504 responsive to the electronic addresses associated with the manufacturer (FIG. 47 and FIG. 48).

Of course, other topologies of system 548 are possible, for instance, capsule design tracking server 550 can in fact constitute several computers that are linked together in a network or be a virtual machine in a cloud computing context. As such, the exemplary topology shown in FIG. 47 merely serves to describe the features of an embodiment of the present disclosure in a manner that will be readily understood to one of skill in the art.

Referring to FIG. 48, in typical embodiments, a capsule design tracking server 550 comprises one or more computers. For purposes of illustration in FIG. 48, the capsule design tracking server 550 is represented as a single computer that includes all of the functionality of the capsule design tracking server 550. However, the disclosure is not so limited. The functionality of the capsule design tracking server 550 may be spread across any number of networked computers and/or reside on each of several networked computers and/or by hosted on one or more virtual machines at a remote location accessible across the communications network 506. One of skill in the art will appreciate that a wide array of different computer topologies is possible for the capsule design tracking server 550 and all such topologies are within the scope of the present disclosure.

Turning to FIG. 48 with the foregoing in mind, an exemplary capsule design tracking server 550 comprises one or more processing units (CPU's) 574, a network or other communications interface 584, a memory 592 (e.g., random access memory), one or more magnetic disk storage and/or persistent devices 590 optionally accessed by one or more controllers 588, one or more communication busses 512 for interconnecting the aforementioned components, and a power supply 576 for powering the aforementioned components. Data in memory 592 can be seamlessly shared with non-volatile memory 590 using known computing techniques such as caching. Memory 592 and/or memory 590 can include mass storage that is remotely located with respect to the central processing unit(s) 574. In other words, some data stored in memory 592 and/or memory 590 may in fact be hosted on computers that are external to capsule design tracking server 550 but that can be electronically accessed by the capsule design tracking server over an Internet, intranet, or other form of network or electronic cable (illustrated as element 506 in FIG. 48) using network interface 584.

The memory 592 of capsule design tracking server 550 stores:
an operating system 602 that includes procedures for handling various basic system services;
an application module 604 for distributing an application to a plurality of subjects designing a tailored capsule;
a questionnaire 606 that includes a plurality of questions, each respective question 600 in the plurality of questions (i) is associated with a corresponding condition 608 in a plurality of conditions associated with the capsule design and (ii) comprises an affordance 610 that is configured to allow the subject to select between a low value and a high value to indicate a degree to which the subject presently associates with the corresponding condition;
a subject data store 614, the subject data store comprising a respective record of each corresponding subject 616 monitored by the disclosed systems, each respective record including (i) a questionnaire response history 618 from the corresponding subject, (ii) one or more electronic addresses 620 associated with the corresponding subject, and (iii) an optional unique subject identifier 624 associated with the corresponding subject for the corresponding subject.

In some implementations, one or more of the above identified data elements or modules of the capsule design tracking server 550 are stored in one or more of the previously described memory devices, and correspond to a set of instructions for performing a function described above. The above identified data, modules or programs (e.g., sets of instructions) need not be implemented as separate software programs, procedures or modules, and thus various subsets of these modules may be combined or otherwise re-arranged in various implementations. In some implementations, the memory 592 and/or 590 optionally stores a subset of the modules and data structures identified above. Furthermore, in some embodiments the memory 592 and/or 606 stores additional modules and data structures not described above.

According to an exemplary embodiment of the present disclosure, a user interface capable of designing a custom, single dosage capsule tailored to the user is proposed. A method of providing the present user interface comprises, at an internet-enabled device with a display, running an application on the internet-enabled device that provides a questionnaire 606 to a user within the application regarding a capsule. The questionnaire comprises a plurality of questions 600. The plurality of questions 600 comprise one or more biometric parameters associated with the user, one or more physician recommendations associated with the user, one or more personal health needs associated with the user, one or more goals associated with the user, and/or one or more nutritional requirements associated with the user of the electronic device. A response to the questionnaire is received 606. The plurality of response to the questionnaire 606 are stored in a date store 614 associated with the user of the electronic device. Responsive to a capsule request from the user, a personalized capsule is designed responsive to the plurality of responses. The designing determines a modified-release schedule and an external capsule shape from all or a portion of the plurality of responses. In some embodiments, the internet-enabled device is a tablet or smart phone. Also, in a further embodiment, the plurality of questions 600 in the questionnaire 606 includes a dietary query, a self-involvement query, and/or an end goal query.

In some embodiments, a non-transitory computer readable storage medium is provided. The non-transitory computer readable storage medium stores instructions, which when executed by a first Internet-enabled device, causes the first Internet-enabled device to perform the above method. In further embodiments, an Internet-enabled computer system comprises one or more processors, memory, and one or more programs stored in the memory for execution by the one or more processors. The one or more programs comprise instructions for performing the above method.

In some embodiments of the present disclosure, a first question in a plurality of questions is associated with a first accordance that is configured to allow the user to select between different values for the respective question. In another embodiment, the user inputs information from their personal computer, a web application, a mobile application, or from a designated terminal. In a further embodiment, the instructions are in the form of a video, a picture, a document, or a uniform resource location link to a document.

In some embodiments, the user selects the final quantities of material in the capsule, or selects which of a plurality of compartments have a modified release schedule or immediate release schedule; however, the present disclosure is not limited thereto. For instance, in another embodiment, the final quantities of material are selected from a predetermined algorithm, or the algorithm selects which of a plurality of compartments have a modified release schedule or immediate release schedule.

In some embodiments, the questionnaire is provided on a reoccurring basis, for instance between capsule design orders or refilling of a previous order. In some embodiments, the questionnaire is provided as a user feedback questionnaire after a first design order is fulfilled.

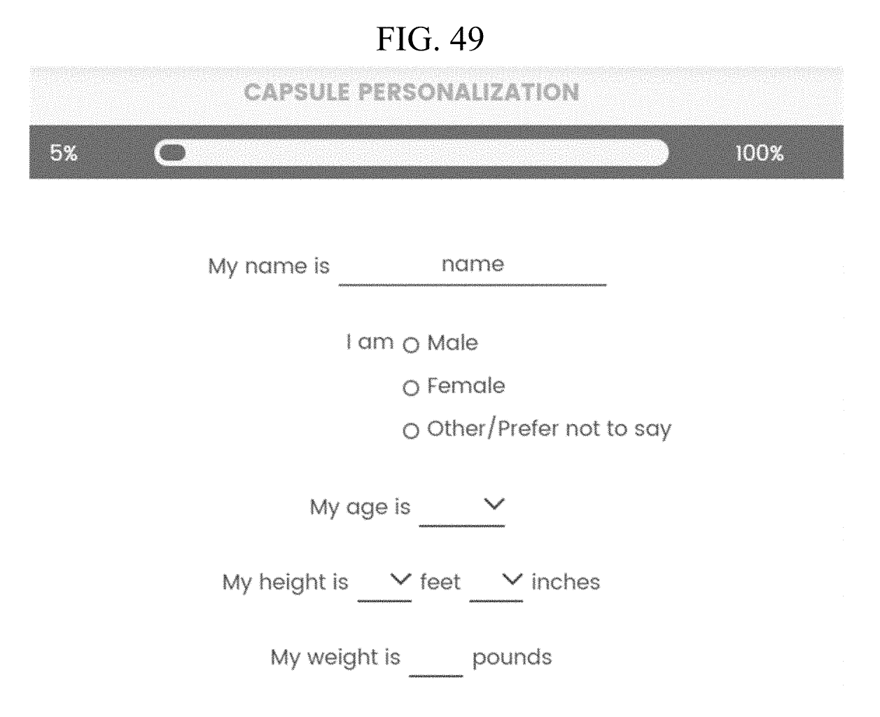

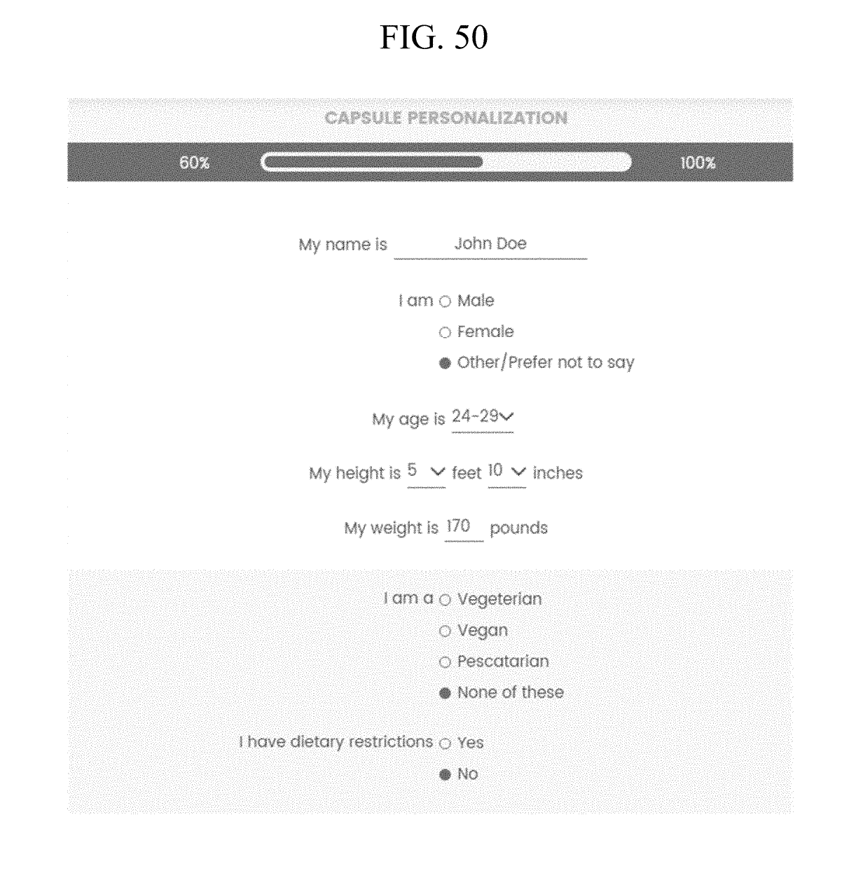

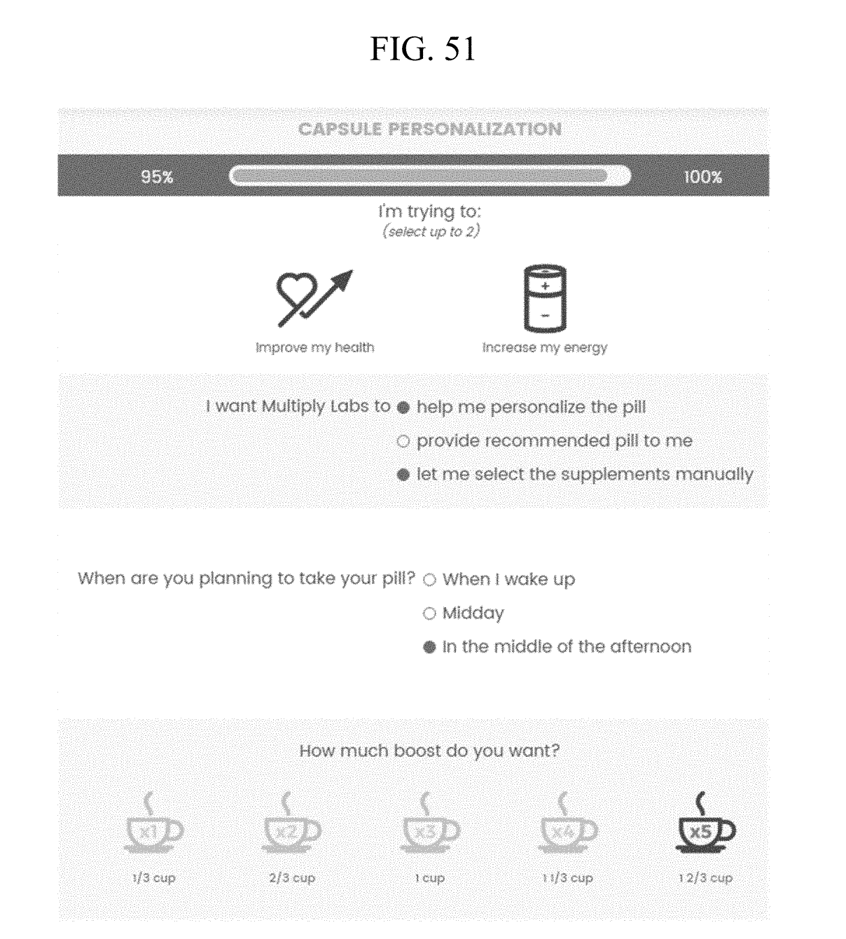

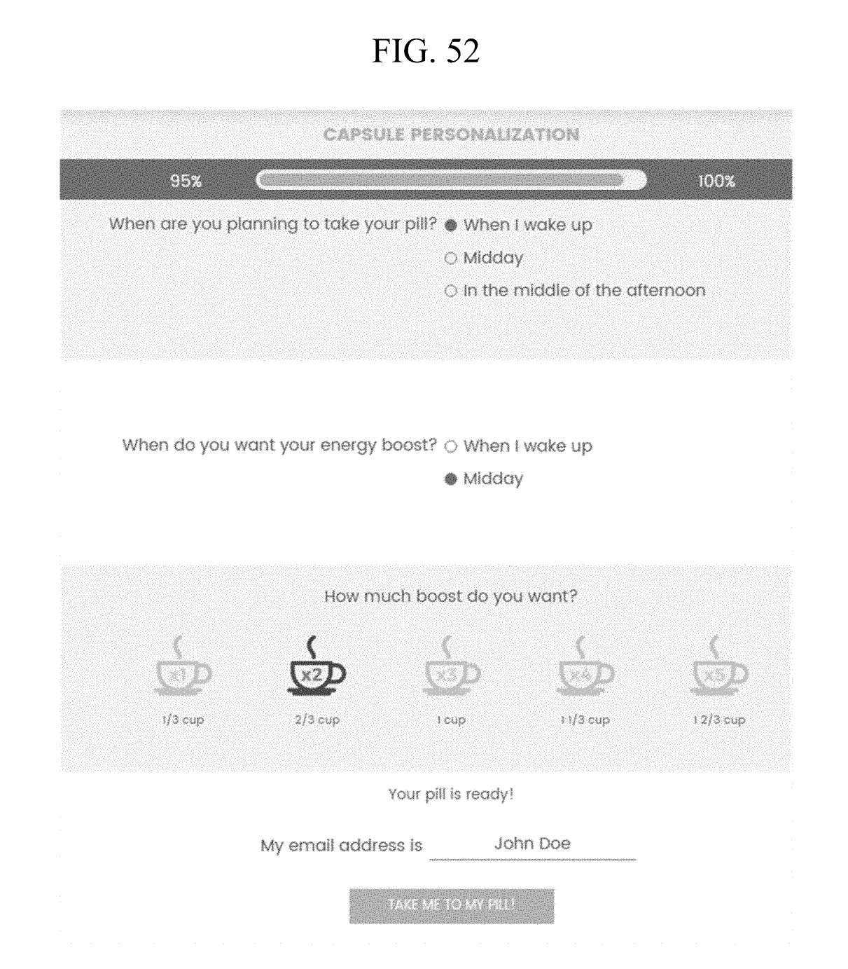

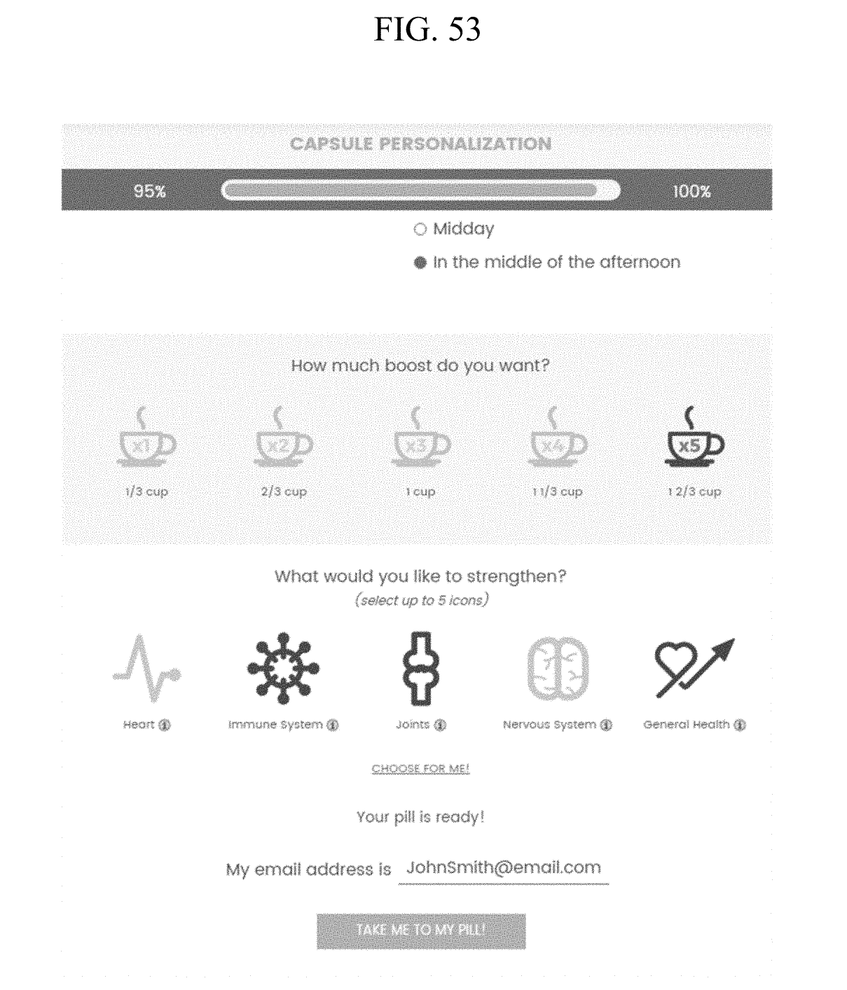

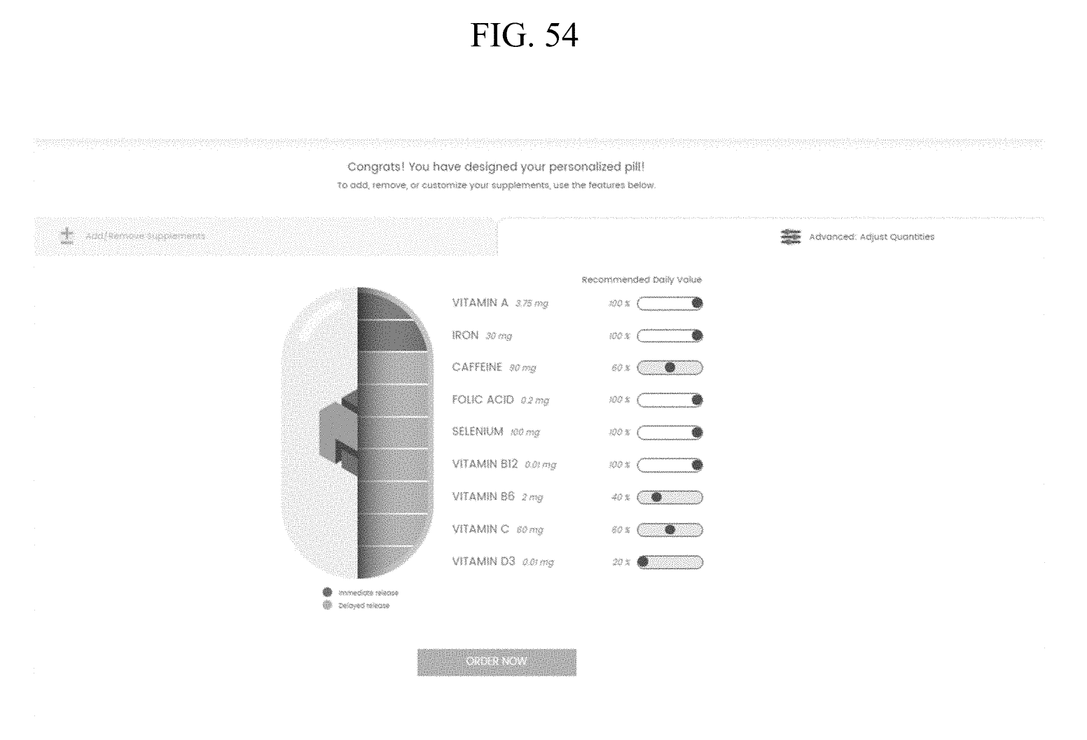

Referring to FIG. 49 to FIG. 54, illustrations of the user interface are provided according to an exemplary embodiment of the present disclosure. The questionnaire 606 comprises a plurality of questions 600. Each respective question in the plurality of questions (i) is associated with a corresponding condition in a plurality of conditions (e.g., fatigue) and (ii) comprises an affordance (e.g., a slide bar) that is configured to allow the subject to select between a low value and a high value to indicate a degree to which the subject presently associates with the corresponding condition.

Accordingly, a user interface according to an exemplary embodiment of the present disclosure achieves the advantages of allowing a user to design a tailored single dose capsule.

For convenience in explanation and accurate definition in the appended claims, the terms “upper”, “lower”, “up”, “down”, “upwards”, “downwards”, “inner”, “outer”, “inside”, “outside”, “inwardly”, “outwardly”, “interior”, “exterior”, “front”, “rear”, “back”, “forwards”, and “backwards” are used to describe features of the exemplary embodiments with reference to the positions of such features as displayed in the figures.

The foregoing descriptions of specific exemplary embodiments of the present invention have been presented for purposes of illustration and description. They are not intended to be exhaustive or to limit the invention to the precise forms disclosed, and obviously many modifications and variations are possible in light of the above teachings. The exemplary embodiments were chosen and described in order to explain certain principles of the invention and their practical application, to thereby enable others skilled in the art to make and utilize various alternatives and modifications thereof. It is intended that the scope of the invention be defined by the Claims appended hereto and their equivalents.

What is claimed is:

1. A filament extruder, attached to a three-dimensional printer, that is configured to lay a plurality of filament solutions on a target surface, the filament extruder comprising:
a plurality of extruder sets, wherein each extruder set in the plurality of extruder sets comprises a first extruder and a second extruder disposed adjacent to the first extruder, wherein each of the first extruder and the second extruder comprises an extruder portion, the extruder portion comprising:
a first insert and a second insert, wherein the first insert and the second insert combine to form a channel, wherein the channel receives and guides a filament solution in the plurality of filament solutions, and
a needle tube that receives the filament solution from the channel of the extruder portion and lays the filament solution on a portion of the target surface;
a hot end portion comprising:
a heater block that encompasses at least a portion of each needle tube of each extruder set in the plurality of extruder sets, and
a heater disposed interposing between the needle tube of the first extruder and the needle tube of the second extruder of each extruder set in the plurality of extruder sets, wherein the heater is configured to melt each filament solution in the plurality of filament solutions; and
a shroud housing the hot end portion.

2. The filament extruder according to claim 1, wherein the extruder portion of each of the first extruder and the second extruder of each extruder set in the plurality of extruder sets further comprises:
a first plate and a second plate configured to hold the first insert and the second insert respectively, wherein the first plate and the second plate are removeably coupled to form a first housing when combined;
a pinch wheel that is proximate to a first side of a first opening of the channel;
a bearing proximate to a second side of the first opening of the channel;
a lever coupled to the bearing, wherein the lever is disposable between (i) a first position in which the lever drives the bearing against the filament solution that is between the bearing and the pinch wheel, thereby causing the filament solution to traverse the channel to the needle tube and (ii) a second position in which the bearing is disengaged from the pinch wheel and the filament solution, thereby preventing the filament solution from traversing the channel to the needle tube; and
a spring that actuates the lever between the first position and the second position.

3. The filament extruder according to claim 2, each extruder set in the plurality of extruder sets further comprises:
a spacer disposed interposing between the first extruder and the second extruder, wherein the spacer is configured to reduce a distance between the first plate and the second plate of each of the first extruder and the second extruder in each extruder set.

4. The filament extruder according to claim 2, wherein each extruder set in the plurality of extruder sets further comprises:
a heat sink disposed on either the first plate or the second plate of one of the first extruder or the second extruder, and
a fan configured to draw air across the first extruder and the second extruder towards the heat sink.

5. The filament extruder according to claim 2, wherein the first plate and the second plate of each of the first extruder and the second extruder of each extruder set in the plurality of extruder sets further comprises a plurality of ventilation holes.

6. The filament extruder according to claim 2, wherein the pinch wheel of the first extruder and the pinch wheel of the second extruder of each extruder set are integrally combined to form a double-pinch wheel.

7. The filament extruder according to claim 1, wherein the heater block further comprises:
a first portion that encompasses at least a portion of each needle tube of each extruder set in the plurality of extruder set, and
a second portion that is removeably coupled to the first portion of the heater block, wherein
the first portion and the second portion of the heater block combine and form a second housing to accommodate the heater of the hot end portion.

8. The filament extruder according to claim 1, wherein the heater block further comprises a first gap configured to restrict a movement of the heater when a force is applied to the first gap.

9. The filament extruder according to claim 1, wherein the heater block further comprises one or more second gaps, wherein each second gap in the one or more second gaps is associated with a corresponding needle tube, the second gap being configured to restrict a movement of the corresponding needle tube when a force is applied to the second gap.

10. The filament extruder according to claim 1, wherein the needle tube of each of the first extruder and the second extruder of each extruder set in the plurality of extruder sets further comprises a nozzle disposed at an outlet of the needle tube.

11. The filament extruder according to claim 2, wherein the needle tube of each of the first extruder and the second extruder of each extruder set in the plurality of extruder sets further comprises a stopper that restricts a vertical movement of the needle tube.

12. The filament extruder according to claim 11, wherein the needle tube of each of the first extruder and the second extruder of each extruder set in the plurality of extruder sets further comprises a groove that accommodates the stopper.

13. The filament extruder according to claim 1, wherein the channel of each of the first extruder and the second extruder of each extruder set in the plurality of extruder sets is formed with a funnel first opening.

14. The filament extruder according to claim 2, wherein the pinch wheel of each extruder portion, the bearing of each extruder portion, the first insert of each extruder portion, the second insert of each extruder portion, the needle tube of each extruder portion, and the shroud are formed of 316 stainless steel.

15. The filament extruder according to claim 1, wherein each component of the filament extruder is removeably coupled, wherein the entire filament extruder can be disassembled and reassembled component by component.

16. The filament extruder according to claim 1, wherein each filament solution in the plurality of filament solutions is a same material.

17. The filament extruder according to claim 1, wherein the plurality of extruder sets comprises one extruder set.

18. The filament extruder according to claim 1, wherein the channel is formed with a funnel first opening.

19. A filament extruder, attached to a three-dimensional printer, that is configured to lay a first filament solution on a first portion of a target surface and a second filament solution on a second portion of the target surface, the filament extruder comprising:
an extruder set comprising a first extruder and a second extruder disposed adjacent to the first extruder, wherein each of the first extruder and the second extruder comprises an extruder portion, the extruder portion comprising:
a first insert and a second insert, wherein the first insert and the second insert combine to form a channel, wherein the channel receives and guides a respective filament solution,
a first plate and a second plate configured to hold the first insert and the second insert respectively, wherein the first plate and the second plate are removably coupled to form a housing when combined,
a pinch wheel that is proximate to a first side of a first opening of the channel,
a needle tube that receives the filament solution from the channel of the extruder portion and lays the filament solution on a respective portion of the target surface,
a bearing proximate to a second side of the first opening of the channel,
a lever coupled to the bearing, wherein the lever is disposable between (i) a first position in which the lever drives the bearing against the filament solution that is between the bearing and the pinch wheel, thereby causing the filament solution to traverse the channel to the needle tube and (ii) a second position in which the bearing is disengaged from the pinch wheel and the filament solution, thereby preventing the filament solution from traversing the channel to the needle tube, and
a spring that actuates the lever between the first position and the second position;
a spacer disposed interposing between the first extruder and the second extruder of the extruder set, wherein the spacer is configured to reduce a distance between the first plate and the second plate of each of the first extruder and the second extruder of the extruder set;
a hot end portion comprising:
a heater block that encompasses at least a portion of each needle tube of the first extruder and the second extruder of the extruder set, and
a heater disposed interposing between the needle tube of the first extruder and the needle tube of the second extruder of the extruder set, wherein the heater is configured to melt the first filament solution and the second filament solution; and
a shroud housing the hot end portion.

20. A filament extruder, attached to a three-dimensional printer, that is configured to a plurality of filament solutions a target surface the filament extruder comprising:
a plurality of extruders, wherein each extruder in the plurality of extruders is disposed adjacent to another extruder in the plurality of extruders, and wherein each extruder in the plurality of extruder comprises an extruder portion, the extruder portion comprising:
a first insert and a second insert, wherein the first insert and the second insert combine to form a channel, wherein the channel receives and guides a respective filament solution,
a first plate and a second plate configured to hold the first insert and the second insert respectively, wherein the first plate and the second plate are removeably coupled to form a housing when combined,
a pinch wheel that is proximate to a first side of a first opening of the channel,
a needle tube that receives the filament solution from the channel of the extruder portion and lays the filament solution on a respective portion of the target surface,
a bearing proximate to a second side of the first opening of the channel,
a lever coupled to the bearing, wherein the lever is disposable between (i) a first position in which the lever drives the bearing against the filament solution that is between the bearing and the pinch wheel, thereby causing the filament solution to traverse the channel to the needle tube and (ii) a second position in which the bearing is disengaged from the pinch wheel and the filament solution, thereby preventing the filament solution from traversing the channel to the needle tube, and
a spring that actuates the lever between the first position and the second position;
a spacer disposed interposing between each extruder in the plurality of extruders and the adjacent extruder of the plurality of extruders, wherein the spacer is configured to reduce a distance between the first plate and the second plate of each extruder in the plurality of extruders and the adjacent extruder in the plurality of extruders;
a hot end portion comprising:
a heater block that encompasses at least a portion of each needle tube of the first extruder and the second extruder of the extruder set, and
a heater disposed proximate to the needle tube of each extruder in the plurality of extruders, wherein the heater is configured to melt the filament solution of the corresponding extruder in the plurality of extruders; and
a shroud housing the hot end portion
